(19) United States
(12) Patent Application Publication
(10) Pub. No.: US 2008/0118570 A1
(43) Pub. Date: May 22, 2008

(54) POLYMER COATED DRUG-ION EXCHANGE RESINS AND METHODS

(76) Inventors: Zhi Liu, Mundelein, IL (US); Bei Chen, Buffalo Grove, IL (US); Danchen Gao, Chicago, IL (US); Mukti S. Rao, Mundelein, IL (US); Arunya Usayapant, Mundelein, IL (US)

Correspondence Address: WINSTON & STRAWN LLP;PATENT DEPARTMENT, 1700 K STREET, N.W., WASHINGTON, DC, 20006, US

(21) Appl. No.: 11/561,723

(22) Filed: Nov. 20, 2006

(51) Int. Cl. A61K 9/16; A61K 47/48; A61K 31/439; A61K 31/216; A61K 31/4355; A61K 31/137

(52) U.S. Cl. 424/490; 514/772.3; 514/289; 514/534; 514/282; 514/654

(57) ABSTRACT

Included are compositions, and methods of making, coated controlled-release drug and ion exchange resin form complexes. Methacrylate coatings, which can be free of plasticizers particularly with Eudragit® NE type polymer, are preferred to enhance the control of drug release from the drug-resin complexes. Liquid formulations including the coated resin forms and a chelating agent to inhibit degradation are also included.

### FIELD OF THE INVENTION

The invention relates to pharmaceutical compositions that include controlled-release, methacrylate polymer coated drug-ion exchange resin, where the coating is substantially free of plasticizer. Methods of preparing such coated drug/resin complexes and solid and/or suspended formulations containing the same are also included.

### BACKGROUND OF THE INVENTION

The technology of forming liquid controlled-release formulations of ionic drugs is generally known. This is conventionally achieved with one or more ionic drugs (interchangeably referred to herein as an “active pharmaceutical ingredient”) that are first ionically bound using their positive charge to particulate ion-exchange resins (interchangeably referred to herein as “resins,” “ionic exchange resin forms,” “resin beads,” or “beads”), such as sodium polystyrene sulfonate Amberlite® IRP-69, USP (manufactured by Rohm Haas of Germany). The resonate (interchangeably referred to herein as the “drug-resin complex” or “drug/resin complex”) in some instances can be further coated with a thin film of a suitable polymer, such as, for example, ethylcellulose, in Wurster process, which is a process that ensures uniform coating of particulates. The function of the polymer film is to serve as a diffusion barrier to reduce the drug release rate when the drug is freed from the resin. The coated beads are conventionally suspended in a viscous vehicle, typically in a ready-to-use suspension form. As used herein, a “pharmaceutical suspension” is a coarse dispersion in which insoluble solid particles are generally dispersed in a liquid medium.

Drug release from the resin, in vivo or in vitro, is triggered by diffusion of external electrolyte solution into the drug resin complex. The electrolyte is present in normal physiological conditions in vivo, or in a properly designed in vitro dissolution media containing such electrolyte. Generally, the drug molecules, ionically bound to resin, will be replaced by the external electrolyte. Then, the unbound drug passes through the coated polymer film by the diffusion mechanism.

While ionic exchange resin forms allow some degree of controlled drug release, more precise and higher levels of control are not available in conventional drug compositions. That is, controlled release drugs should have a mechanism to deliver the drug over a predetermined period of time, and after the end of that predetermined period of time, the drug, preferably in a drug resin complex, should completely dissolve, thereby evacuating the drug from the drug resin and/or allowing complete absorption of the drug in the subject within the predetermined amount of time. It is therefore desirable to develop drugs with enhanced control of drug release.

Coatings are generally known as one method of affecting the release profile of a drug. The type and amount of the coating is typically the primary regulator of the release rate of the drug. PennKinetic® technology, for example, was developed by Pennwalt and involves the use of ion exchange resins in combination with diffusion-controlling membranes to create controlled-release liquid dosage forms. Several liquid products have incorporated this sustained release coating technology, including, for example, sustained release Delsym® brand (manufactured by UCB, Inc. of New York, USA), which is ethylcellulose coated dextromethorphan polistirex; sustained release Tussionex® brand (manufactured by UCB, Inc. of New York, USA), which is a combination of ethylcellulose coated hydrocodone polistirex and uncoated chlorpheniramine polistirex; and sustained release Codeprex® brand (manufactured by UCB, Inc. of New York, USA), which is comprised of ethylcellulose coated codeine polistirex and uncoated chlorpheniramine polistirex.

The above-mentioned products utilize a solvent-based coating. Aqueous coating processes, which were subsequently invented, are additional alternatives in forming sustained release products. There are numerous advantages of aqueous coating over solvent coating, including eliminating flammable and sometimes toxic organic solvents, improving safety, eliminating down stream-solvent incarnation, and diminishing the issue of residual organic solvent in the finished product. Many regulatory restrictions, such as those stemming from environmental and toxicological concerns associated with organic solvent-based process in solvent-based systems, have accelerated the transition to aqueous-based coating systems.

Despite the movement towards aqueous-based systems, the FDA has yet to approve an oral liquid controlled release product based on aqueous coating using PennKinetic® technology. The aqueous process is typically used in the field of coating pellets and tablets, which are generally much bigger in size than the resin substrates used in PennKinetic® technology. Coating of small size particles has been proven much more technically challenging.

In aqueous coatings, there have been different classifications of polymers depending upon their solubility, permeability, and disintegration properties. Two most widely used polymers are acrylic polymers, such as the Eudragit® series of polymers (manufactured by Degussa/Rohm Haas of Germany) and cellulose-based polymers, including Aquacoat® (by FMC Biopolymer of Pennsylvania, USA) and Surelease® (by Colorcon, Inc. of Pennsylvania, USA).

The influence of formulation factors on the stability and performance of film-coated dosage forms has been studied. The drug release rate has been shown to be influenced by the hydrophilic and lipophilic properties, and the amounts, of polymers and plasticizers in the film coating, and the type and level of anti-adherent, that is applied to the substrate. Aqueous polymers typically require plasticizer to facilitate the coating formation process. The plasticizer serves several purposes including increasing the elasticity of the film and preventing crack formulation in films from mechanical stresses.

U.S. Publication No. 2005/0181050 to Hirsch et al. discloses the use of Eudragit® polymers of various types coating drug loaded ion exchange resin forms. The drug resin complexes are treated prior to the coating process with an impregnating agent such as polyethylene glycol. The publication discloses that the coating composition may include conventional additives, such as plasticizers, normally in amount from 10 to 50 weight percent relative to the dry weight of the polymer.

Thus, there is an unmet need in the art for compositions and methods to increase the control of drug release from drug-resin complexes. Specifically, there is an unmet need in the art for compositions of coated ion exchange resin forms that release drugs in a controlled manner, and methods for making the same. Moreover, there is an unmet need in the art to coat small size particles in an aqueous manner.

### SUMMARY OF THE INVENTION

The present invention can advantageously meet one or more of these unmet needs of the art by providing the inventive compositions and methods. The invention relates to a method of making a coated drug/resin complex by providing a drug/resin complex having an outer surface and including a plurality of resin beads and a therapeutically effective drug component releasably associated therewith, applying a coating layer including a methacrylate polymer, preferably a neutral copolymer of polyethyl acrylate and polymethyl methacrylate, more preferably having a ratio of polymethyl acrylate to polymethyl methacrylate from about 0.5:1 to 10:1, and even more preferably Eudragit® NE polymer, over a portion of, preferably substantially entirely around, the outer surface of the complex in an amount sufficient to control the release rate of the drug component so as to form a coated drug/resin complex, wherein the coating layer is at least substantially free of plasticizer and forms a flexible material that resists cracking. The drug component preferably includes hydrocodone, oxybutynin, dextromethorphan, fluoxetine, metoprolol, venlafaxine, buproprion, doxycycline, or verapamil, or a salt thereof, or any combination thereof.

The invention also relates to coated drug/resin complexes that include a plurality of dry ion exchange resin forms complexed with a drug component to form a drug/resin complex, and a substantially plasticizer-free coating layer having a thickness that includes a sufficient amount of methacrylate polymer applied at least substantially around a portion of the resin forms to control the release rate of the drug component associated therewith. The coated drug/resin complex is preferably suspended in a pharmaceutically acceptable liquid carrier, which may include a buffering agent, suspending agent, thickening agent, anti-tacking agent, preservative component, sweetening agent, mouth-feel agent, wetting agent or combination thereof.

Yet another aspect of the invention relates to a drug/resin complex including a plurality of dry ion exchange resin forms complexed with a drug component to form a drug/resin complex, and at least about 5 weight percent polyethylene glycol (PEG), based on the complex, that is associated with at least the resin forms to form a PEG-loaded drug/resin complex.

In a further aspect, the invention relates to methods of making a drug/resin complex that includes providing a drug/resin complex having an outer surface and including a plurality of resin beads and a therapeutically effective drug component releasably associated therewith, and contacting a sufficient amount of polyethylene glycol (PEG) with the complex so as to associate the polyethylene glycol therewith to form a PEG-loaded complex having at least about 5 weight percent PEG based on the PEG-loaded complex.

In yet a further aspect, the invention relates to a method of manufacturing a pharmaceutical composition by providing a plurality of ion exchange resin forms complexed with a drug component to form a drug/resin complex, providing a coating to the drug/resin complex, and suspending the coated resin forms in a liquid formulation that includes a sufficient amount of chelating agent and/or antioxidant to inhibit or prevent degradation of the drug-resin complex over a shelf life of the composition. In a preferred embodiment, the chelating agent includes EDTA, citric acid, or a salt thereof, or any combination thereof. In a preferred embodiment, the antioxidant component includes ascorbic acid, or a salt thereof, and tocopherol, sorbic acid, retinol, butylated hydroxyanisole (BHA), butylated hydroxytoluene (BHT), propyl gallate, or any combination thereof.

The invention also encompasses a coated drug/resin complex that includes a plurality of dry, ion exchange resin forms complexed with a drug component to form a drug/resin complex, and a neutral coating layer having a thickness that includes a sufficient amount of methacrylate polymer applied at least substantially around a portion of the resin forms to control the release rate of the drug component associated therewith. In some embodiments, the sufficient amount of the methacrylate coating is greater than about 5 weight percent, and more preferably between about 7 weight percent to 40 weight percent, based on the coated drug/resin complex.

In a preferred embodiment, the coated drug/resin complex is made by a process that includes providing a drug/resin complex having an outer surface and including a plurality of resin beads and a therapeutically effective drug component releasably associated therewith, and applying a coating layer including a methacrylate polymer over a portion of the outer surface of the complex in an amount sufficient to control the release rate of the drug component so as to form a coated drug/resin complex, wherein the coating layer is neutral and forms a coat that is sufficiently flexible to resist or avoid cracking.

Any of the embodiments illustrated herein stand independently, and any features or embodiments may be combined in any way, unless expressly excluded, to achieve a preferred embodiment. Additional advantages and embodiments of the invention will also become more apparent to those of ordinary skill in the art upon review of the teachings of the present application.

### BRIEF DESCRIPTION OF DRAWINGS

Further advantageous features of the present invention will become more apparent with the following detailed description when taken with reference to the accompanying drawings in which:

FIG. 1 is a graph depicting the dissolution over time of an oxybutynin ion exchange resin complex, which is coated with varying levels of Eudragit® NE, but in the absence of PEG, in accordance with the invention;

FIG. 2 is a graph depicting the dissolution over time of an oxybutynin ion exchange resin complex, which is coated with varying levels of Eudragit® NE, with 7% PEG by weight of the complex, in accordance with the invention;

FIG. 3 is a graph depicting the dissolution over time of an oxybutynin ion exchange resin complex, which is coated with varying levels of Eudragit® NE, with 14% PEG by weight of the complex, in accordance with the invention;

FIG. 4 is a graph depicting the percent cumulative release of an oxybutynin ion exchange resin complex over time, wherein levels of Eudragit® NE are constant at 15% and levels of PEG are varied, in accordance with the invention

FIG. 5 is a graph depicting the percent cumulative release of a hydrocodone ion exchange resin complex over time, wherein levels of Eudragit® NE are varied and levels of PEG are maintained at 0%, in accordance with the invention;

FIG. 6 is a graph depicting the percent cumulative release of a hydrocodone ion exchange resin complex over time, wherein levels of Eudragit® NE are varied and levels of PEG are maintained at 14% w/w of the composition, in accordance with the invention;

FIG. 7a is a graph depicting the percent cumulative release of a hydrocodone ion exchange resin complex over time, wherein levels of Eudragit® NE are kept at 15% and levels of PEG are varied, in accordance with the invention;

FIG. 7b is a graph depicting the percent cumulative release of a hydrocodone ion exchange resin complex over time, wherein levels of Eudragit® NE are kept at 20% and levels of PEG are varied, in accordance with the invention;

FIG. 7c is a graph depicting the percent cumulative release of a hydrocodone ion exchange resin complex over time, wherein levels of Eudragit® NE are kept at 25% and levels of PEG are varied, in accordance with the invention;

FIG. 8 is a graph depicting the release profile of venlafaxine polistirex over time, wherein levels of Eudragit® NE are varied with PEG being at 8%, in accordance with the invention;

FIG. 9 is a graph depicting the release profile of metoprolol polistirex over time, wherein levels of Eudragit® NE are varied with PEG being at 8%, in accordance with the invention;

FIG. 10 is a graph depicting the release profile of suspensions made from 10% Eudragit® NE and 16% Eudragit® RS/RL (RS to RL ratio being 93 to 7) coated dextromethorphan polistirex suspensions in accelerated stability conditions (40° C.) for up to 8 weeks, in accordance with the invention;

FIG. 11 is a graph depicting the release profile of suspensions made from Eudragit® NE coated hydrocodone polistirex and uncoated chlorpheniramine polistirex in accelerated stability conditions (40° C.) for up to 8 weeks, in accordance with the invention;

FIG. 12 is a graph depicting the release profile of suspensions containing 0.5% w/v citric buffer made from 10% Eudragit® NE (with 7% PEG) coated dextromethorphan polistirex suspensions in accelerated stability conditions (40° C.) for up to 12 weeks, in accordance with the invention; and

FIG. 13 is a graph depicting the release profile of suspensions made from 25% Eudragit® NE (with 14% PEG) coated oxybutynin in accelerated stability conditions (40° C.) for up to 3 months, in accordance with the invention.

### DETAILED DESCRIPTION OF THE PREFERRED EMBODIMENTS

The present invention provides compositions and methods of making the same for providing controlled release of a drug component of one or more pharmaceutically active ingredients. While the invention encompasses both liquid and solid forms, preferably the invention relates to liquid formulations. More specifically, the present invention relates to a complex of a drug and an ion exchange resin form that is typically coated with an aqueous dispersion of a methacrylate polymer, the methacrylate polymer preferably being substantially free, or completely free, of plasticizer, and yet still retaining sufficient flexibility to minimize or avoid cracking of the coating layer. Another aspect of the invention relates to increased loading of polyethylene glycol in association with the drug/resin complex in an amount sufficient to increase the cumulative drug release. The amount of drug release is dependent on the type of drug. Yet another aspect of the invention relates to a stabilizing agent (e.g., one or more chelating agents and/or antioxidants) included in the liquid carrier, in which coated or uncoated drug/resin complexes of the invention can be suspended to provide ready-to-use or reconstitutable liquid formulations.

As used herein, controlled release (also referred to herein as “modified release” or “sustained release”) encompasses drug release characteristics of time, course, and/or location that are chosen to accomplish therapeutic or convenience objectives (e.g., increased patient compliance) not offered by conventional dosage forms, such as solutions, conventional ointments, or promptly dissolving dosage forms. Delayed release, extended release, and pulsatile release and their combinations are the representative types of controlled release. Delayed release encompasses releasing a drug (or drugs) at a time other than promptly after administration, or that begins promptly after administration but ends at a later time. Extended release encompasses release of at least a twofold reduction in dosing frequency, as compared to that drug presented as with immediate release. Pulsatile release mimics a multiple dosing profile without repeated administration and allows at least a twofold reduction in dosing frequency, as compared to immediate release.

The controlled-release, coated drug complexes of the invention advantageously provide more than simply controlled release. The inventive coated complexes typically provide a reproducible amount of drug release. Preferably, the inventive complexes increase the percentage of drug release while minimizing or avoiding residual, undissolved amounts in the resin forms to ensure full and proper dosing.

The drug-resin complex can be formed from any drug for which controlled release is desired in association with any conventional ion exchange resin, as further discussed below. According to the invention, the complex can include one or more layers that encapsulates, i.e., surrounds a core of drug-resin complex forms. Although more than one layer is contemplated, a single coating layer is preferred for ease of manufacturing. Thus, all further discussions herein reference only a single coating layer although the invention covers additional coating layers. The encapsulation can be partial but covers at least half of the surface area of a complex form, preferably enough of the surface area to control the release of the drug from the complex form. More preferably, however, the coating layer substantially completely, or completely, surrounds the core forms. The coating layer can vary in thickness in different locations around a particular form, but preferably the coating layer is of a substantially or entirely uniform thickness around the cores. Coating can slow the rate of dissolution in vitro and in vivo.

The coating materials of general interest are copolymers from the family that includes polymethacrylates (also commonly referred to as “polymeric methacrylates”). The USPNF identifies these compounds under monographs for “ammonio methacrylate copolymer,” “methacrylic acid copolymer,” and “methacrylic acid copolymer dispersion.” Conventionally, some suitable polymethacrylates are commercially available under the trade names Eastacryl® (by Eastman of Tennessee, U.S.A.), Eudragit®, and Kollicoat® (by BASF Pharma of Germany). For complete specifications on polymethacrylates, reference is made to the section entitled “Polymethacrylates” in Rowe, R. et al., The Handbook of Pharmaceutical Excipients, Pharmaceutical Press, Fourth, Edition, 2003, pages 462-468, which is incorporated herein by reference thereto. Generally, methacrylate polymers are known as synthetic cationic, neutral, and anionic polymers of dimethylaminoethyl methacrylates, methacrylic acid, and methacrylic acid esters in varying rations.

Suitable methacrylate polymers that may be in the coating layer of the present invention are those that retain sufficient resilience to minimize or avoid cracking or other damage to the coating layer that might result in diminished uniformity of drug release. The preferred polymers included in the coating layer are Eudragit® acrylic polymers. In certain embodiments of the present invention, particularly with Eudragit® methacrylate polymers, the coating layer is sufficiently elastic to form a coat on the drug resin complex in a layer that is substantially free of plasticizer, or preferably entirely free of plasticizer. That is, the coating layer is substantially plasticizer free but retains its elasticity and structural integrity over a range of temperature and moisture content conditions that occur throughout the life of a pharmaceutical product, i.e., from manufacture, to transport, to storage, to possible reconstitution, through administration. According to the invention, a preferred coating material includes Eudragit® NE 30, which is a neutral copolymer based on poly(ethyl acrylate, methyl methacrylate). Preferably, the ratio of polyethyl acrylate to polymethyl methacrylate in forming the copolymer is about 0.5:1 to 10:1, more preferably about 1:1 to 5:1. In one most preferred embodiment, the ratio is about 1.5:1 to 3:1. An exemplary ratio is about 2:1.

Preferred methacrylate polymers include Eudragit® NE 30D and Eudragit® NE 40D (“NE” indicates neutral Lester and “D” indicates dispersion). As used herein, Eudragit® NE 30D and Eudragit® NE 40D is collectively referred to as “Eudragit® NE.” The generic chemical formula of such polymers is

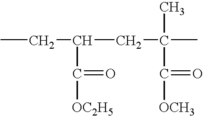

having the Chemical Abstract Service (CAS) Registry No. 9010-88-2.

Any suitable weight percentage methacrylate polymer can be included in the coating of the present invention. The dry weight content of the preferred polymer may range from about 1 to 45%, preferably from about 5 to 42%, and more preferably from about 10 to 40%, by weight of an aqueous coating dispersion. Eudragit® NE 30D, for example, is preferably applied as an aqueous dispersion with a polymer dry weight content of 30%, although as noted above other dry weight contents are suitable. Another exemplary commercially available methacrylate polymer contains, e.g., a 40 weight % aqueous dispersion. Other neutral polymer materials having differing weights can be included so long as they possess coat-forming capability, preferably to form coatings that are at least substantially free of plasticizer.

The methacrylate polymers of the invention are typically swellable and permeable. Generally, polymer dispersions were obtained by emulsion polymerization, although any suitable manner of preparing or applying suitable polymethyl methacrylate polymers to the drug/resin complex forms may be used. In this process, the polymer is precipitated from the monomer units emulsified in water in the form of water-insoluble, submicroscopic latex particles typically ranging from about 0.01 to 1 mm in diameter. The resultant polymer dispersions typically have high solids contents of about 30 to 40%, are very low in viscosity, and are easy to further process.

It has now been found that the preferred polymethyl methacrylate polymers, especially Eudragit® NE 30D, in a coating as a controlled release layer does not necessarily require the use of plasticizer. Accordingly, in embodiments of the present invention without plasticizer, the preferred coating polymer includes Eudragit® NE 30D, generic or trade versions thereof, or methacrylates of similar chemical, physical, functional, and structural properties. Without being bound by any theory, the plasticizer appears to be unnecessary in controlled release coatings of Eudragit® NE 30D because of its low film formation temperature and great elasticity of the layer material made of Eudragit® NE 30D. Surprisingly, these preferred Eudragit® NE polymers provide superior coatings compared to Eudragit® RS or RL polymers, or a combination of RS/RL polymers.

In one embodiment, the invention includes one or more coating layers formed so as to include a substantially or entirely neutral material. Preferably, the coating layer(s) will be at least substantially free, and more preferably, entirely free of charged materials, which can cause coating, stability, or other problems in the formulation of various controlled-release products. Preferably, a substantially or entirely neutral coating layer will be disposed directly adjacent the drug/resin complex. For example, all coating layers can be neutral or one or more intermediate or outermost coating layers can be charged when the coating layer adjacent the complex is substantially or entirely neutral. It would be understood to one of ordinary skill in the art that the neutral charge coating layer may include two or more polymers that are of non-neutral charges that are each present in an amounts sufficient to provide a neutral layer, provided that each is sufficiently mixed or otherwise dispersed to also provide a neutral local charge.

The coating material of polymethyl methacrylate polymer, along with any suitable optional additives, is used to coat the ion exchange resin forms. These forms preferably have an average size less than about 100 μm. In some embodiments of the present invention, aqueous-based Eudragit® NE 30D is used to coat ion exchange resins of about 30 μm to 300 μm in size. In one preferred embodiment, the resin size is about 45 μm to 200 μm.

As use herein, the term “substantially surrounds” (or “substantially surrounded”) means enough coating material so that the release rate is controlled. It would be understood to one of ordinary skill in the art that release rate is controlled by a combination of factors, including thickness size of the coating, coating amount, and percentage of ion exchange resin form surface covered, encompassed together. It would also be known to one of ordinary skill in the art how to adjust these factors to control the release rate. In a preferred embodiment, the ion exchange resin forms can be substantially surrounded, or more preferably fully enclosed, encapsulated and/or coated by the coating layer. In a more preferred embodiment, the coating is disposed or provided over the drug/resin forms in a substantially or entirely uniform thickness. Other embodiments include non-uniform thicknesses. Additionally, although any achievable shape may be used, in preferred embodiments the coated ion resin complex may be an irregular shape, e.g., non-spherical. Preferably, the portion of the outer surface of the complexes that is coated is substantially all, more preferably all, of a plurality of the drug/resin complex. It should be understood that preferably all of the complexes are coated, however, the invention encompasses embodiments where some of the complexes are not as fully coated as others.

The coating formulations of the present invention are preferably capable of producing a strong, continuous layer that is substantially smooth, capable of supporting pigments and other suitable coating additives, non-toxic, inert, and/or tack-free. The dissolution profile of the coated complex may also be modified, for example, by increasing or decreasing the thickness of the coating.

Any procedure that provides a continuous layer on each particle of drug-resin complex without significant agglomeration of particles may be used. Coating procedures known in the pharmaceutical art including, but not limited to, fluid bed coating processes and microencapsulation may be used to obtain suitable coatings on the drug/resin complex forms. Due to the tackiness of certain polymeric materials that may be included in the coating, it is preferred to coat the drug/resin complex forms at a temperature sufficiently below the minimal film formation temperature (MFT) of the materials selected for coating to minimize or avoid tackiness that can inhibit or prevent smooth coatings or coatings of substantially uniform thicknesses. Typically, a coating temperature of at least about 2° C., preferably at least about 5° C., and more preferably at least about 10° C., below the MFT is selected. In general, it is preferable to coat the drug/resin complex forms at temperatures of no more than about 38° C., preferably no more than about 25° C., and more preferably at or even below room temperature. The MFT of Eudragit® NE 30D is much closer to room temperature compared to other polymethyl methacrylate polymers, and other such polymers having a comparable MFT to Eudragit® NE 30D may also be included as or in the coating material.

The coating material to provide the layer disposed over a plurality of the drug/resin complex forms preferably includes a sufficient amount of anti-sticking agent to minimize or avoid tacking of the coating, which preferably appears uniform as seen in optical microscopy and SEM. Any suitable anti-tacking agent available to those of ordinary skill in the art may be used according to the invention. A preferred anti-tacking agent of the present invention includes glyceryl monostearate (GMS), magnesium aluminum silicate, talc, and any combination thereof. The anti-tacking agent may be present in any suitable amount, preferably from about 0.1 weight percent to 15 weight percent, more preferably from about 1 weight percent to 10 weight percent. The specific percentage may depend on which component(s) are included within the anti-tacking agent. Such an anti-tacking agent, if present, may be present in a conventional amount. In an exemplary embodiment, 6 weight percent talc may be added to the coated beads. In another embodiment, magnesium aluminum silicate at 10% weight percent may be added to the coated beads.

The anti-tacking agent can be applied in any suitable manner available to those of ordinary skill in the art. In one embodiment, the anti-tacking agent can be included with the coating material for application to the beads. Preferably, the anti-tacking agent is either homogenized (e.g., GMS) in water or mixed with water (e.g., talc), or both, before being combined with the coating formulation. Preferably, the anti-tacking agent and other coating material are mixed sufficiently to provide a substantially uniform dispersion so the anti-tacking agent is dispersed throughout the coating material. Following this combination, the coating material is applied to the beads. In one embodiment, the coating material is applied to the beads and an anti-tacking agent is then applied to the beads, e.g., through dusting, sifting, spraying, or other suitable application method. Of course, other suitable tacking agents and amounts for use in the present invention may be readily determined known to those of ordinary skill in the art. Preferably, the same or a different anti-tacking agent can be applied before or typically with the coating material, and applied to the coated but uncured resin complexes for curing. For example, an anti-tacking agent can be applied to the coating:curing phases in a ratio of about 1:10 to 1:0.5, preferably about 1:6 to 1:1. In one embodiment, the anti-tacking agent as a whole is not identical when applied in the coating material and over the coating material.

The coating is preferably present in an amount sufficient to control, and typically limit, the release rate of the drug component of one or more active pharmaceutical ingredients in the drug/resin complex. Accordingly, in some embodiments, it is usually necessary to overcoat the substrate (i.e., the complex forms) with a sufficient amount of the Eudragit® NE 30D polymer to have greater than about 2 weight percent up to 50 weight percent, preferably from about 5 weight percent to 45 weight percent, and more preferably greater than about 7 weight percent to 40 weight percent, based on the total weight of the coated drug/resin complex. In one exemplary embodiment, the amount of coating is from about 9 weight percent to 35 weight percent of the coated drug/resin complex. The amount of coating may be lesser or greater depending upon the physical properties of the active agent, the desired release rate, and the manner of incorporation of the same, for example. For example, with dextromethorphan, the amount of coating is about 2 weight percent to 30 weight percent. For example, with oxybutynin, the preferred amount of coating may be from about 7 to 20 weight percent (on the basis of the drug/resin complex without PEG impregnation), or about 10 to 30 weight percent where for example 7% or 14% PEG loading has occurred. With hydrocodone, the preferred coating layer(s) may form at least about 10 to 35 weight percent independent of the amount of PEG in the drug/resin complex. For each of venlafaxine and metoprolol, with an 8% PEG impregnation level, the preferred coating amount may be from about 8 to 25 weight percent and 10 to 30 weight percent, respectively.

In preferred embodiments of the invention, however, at least one of the coating layers is substantially free of plasticizer. While any plasticizer may be minimized or avoided, preferably the term “plasticizer” refers to those plasticizers having a charge, e.g., positive or negative. Thus, in one embodiment, the coating layer is preferably substantially, or entirely, free of charged plasticizer.

As used herein, “substantially free” is understood to be less than about 5 weight percent, preferably less than about 2 weight percent, and more preferably less than about 1 weight percent. In a preferred embodiment, the term “substantially free” refers to less than about 0.5 weight percent, preferably less than about 0.25 weight percent. With respect to plasticizer content, these amounts are with reference to the coating weight. As used herein, “completely free,” “entirely free,” or “free” is understood to mean an absence of, less than analytically detectable amounts, the stated characteristic, such as plasticizer.

It has now been discovered that preferred polymethyl methacrylate coating materials can be substantially, or preferably completely, free of plasticizer, which provides numerous advantages in conjunction with a controlled release drug resin complex. In particular, the absence of plasticizer enhances the controlled release properties of the suspended drug-ion exchange resin complexes coated with aqueous coatings, such as the uniformity of release that can be affected by non-uniform distribution of plasticizer in a coating layer.

In another aspect of the invention, polyethylene glycol (PEG) can additionally be chemically bonded or otherwise associated with the ion exchange resin forms and drug complex. This process is commonly referred to as “PEG loading,” and it is preferably achieved before coating the drug complex particles. Suitable pharmaceutical excipients, and their suggested and conventional amounts, including PEG, as used herein, are fully described in Rowe, R. et al., The Handbook of Pharmaceutical Excipients, Pharmaceutical Press, Fourth Edition, 2003, which types and amounts of PEG are incorporated herein by express reference thereto. Some embodiments of the invention use a solid grade PEG, such as PEG 1450, PEG 1540, PEG 1540, PEG 2000, PEG 3000, PEG 3350, PEG 4000, PEG 4600, PEG 8000 or suitable other PEG molecules. In a preferred embodiment, the PEG has a density of about 1.08 g/cm³ and a freezing point from about 30° C. to 70° C. In some embodiments of the present invention, any suitable PEG that is a solid at room temperature can be used for the PEG impregnation (also referred to as PEG loading).

Without being bound by theory, it is believed that the PEG impregnation helps defer and enhance the cumulative dissolution amount of drugs following administration, e.g., the controlled release profile occurs over shorter periods than with a drug/resin complex untreated with PEG. The PEG impregnation is believed to change the dissolution profile of the coating layer to defer and increase the dissolution rate of the drug, which minimizes the amount of drug remaining in the resin at the end of the desired treatment/administration period. Thus, the drug penetrates the coating layer, but the PEG does not affect the efficacy of the drug. In some embodiments, the greater the amount of PEG, the more rapidly the drug is released. In other embodiments, the greater the amount of PEG, the more drug is released and consequently the less remains in the drug/resin complex after a predetermined period of time.

The amount of PEG impregnated into the drug-resin complexes, when optionally included, is preferably sufficient to increase drug dissociation. Preferably, the amount is insufficient to minimize or avoid too great an increase in the speed of drug release. More specifically, in some embodiments, the amount of PEG is greater than 5 weight percent to about 18 weight percent of the drug resin complex, preferably about 7 weight percent to 16 weight percent of the drug resin complex, and more preferably greater than about 12 weight percent of the drug resin complex. The PEG may be loaded as further discussed below.

Preferably, the drug/resin complexes are suspended in a pharmaceutically acceptable liquid carrier to form the liquid pharmaceutical composition. Preferably, the liquid carrier includes various conventional components included in such liquid pharmaceutical compositions, and typically this is an aqueous-based carrier. In particular, the liquid carrier includes or is associated with a stabilizing agent to help stabilize the coated drug component once suspended. A preferred stabilizing agent includes a chelating agent, an antioxidant component, or a combination of chelating agent and/or antioxidant component, which are preferred classes of stabilizing agent for use in accordance with the invention. A chelating agent typically scavenges for trace amounts of metal ions and can stabilize drugs by retarding or reducing oxidation thereof.

Although any suitable chelating agent known to those of ordinary skill in the art may be used, a preferred chelating agent includes one or more of ethylene diaminetetraacetic acid (EDTA), desferrioxamine B, deferoxamine, dithiocarb sodium, penicillamine, pentetate calcium, a sodium salt of pentetic acid, succimer, trientine, nitrilotriacetic acid, trans-diaminocyclohexanetetraacetic acid (DCTA), diethylenetriaminepentaacetic acid, bis(aminoethyl)glycolether-N,N,N′,N′-tetraacetic acid, iminodiacetic acid, citric acid, tartaric acid, fumaric acid, or any salt thereof (of any acids or bases), or a combination thereof. More preferably, the chelating agent is selected to include one or more of citric acid or a salt thereof, or a combination thereof.

Any suitable antioxidant agent available to those of ordinary skill in the art may be used. A preferred antioxidant component includes one or more of ascorbic acid, tocopherol, sorbic acid, retinol, butylated hydroxyanisole (BHA), butylated hydroxytoluene (BHT), propyl gallate, or any salt thereof (of any acids or bases), or a combination thereof. More preferably, the antioxidant component includes ascorbic acid, BHA, BHT, or a combination thereof. In some embodiments, increased drug stabilization can be achieved by using a combination of a chelating agent and antioxidant component. The amount of stabilizing agent should be effective to reduce, preferably substantially, degradation of the drug-resin complex. Typically, this stabilization should be sufficient to minimize or avoid degradation over the shelf life of the liquid pharmaceutical compositions of the invention. In some variations, the stabilizing agent can be present in a concentration of about 0.001% to 20% by weight, preferably from about 0.01% to 10% by weight, and more preferably from about 0.1% to 5% by weight. In a more preferred embodiment, the amount of the stabilizing agent is from about 0.2% to 2.5% by weight for a solid dosage form. In a preferred embodiment, the stabilizing agent is a chelating agent. For a dosage form that is a suspension, the concentration of the chelating agent (e.g., citric-containing buffer) is typically from about 0.1% to 3%, preferably from about 0.25% to about 2%, and most preferably from about 0.4% to 1.5% by weight. As those of ordinary skill in the art will recognize, it may be necessary to adjust the amount of chelating agent upwardly or downwardly depending on various factors including the type of impurities present in the suspension. For example, when an antioxidant component is included, such as BHA or BHT, the amount preferably present might be from about 0.005% to 0.05% by weight.

In a preferred embodiment, citric acid is included in the stabilizing agent. The concentration of the citric buffer is typically from at least about 0.25% to 5%, preferably at least about 0.3% to 4%, and more preferably at least about 0.5% to 3%, of the liquid carrier. In a most preferred embodiment, the amount of citric buffer is about 0.5%. FIG. 12 illustrates the stability at 40° C. of Eudragit® NE coated dextromethorphan polistirex suspension with citric buffer up to 12 weeks. The dissolution of the suspension is relatively unchanged over the test period.

The degradation rate of each drug in a drug-resin complex typically depends on the particular drug, the resin, and other factors such as storage temperature. The rate of degradation is preferably as low as possible. Preferably, a stabilizing agent is present in an amount effective to reduce the amount of degradation of a drug in the drug/resin complex by more than 20% over twelve months of storage at room temperature, relative to an otherwise identical pharmaceutical composition without the stabilizing agent. For instance, if two samples of a drug/resin complex, one treated with stabilizing agent and one untreated, each had 20% by weight of drug at the beginning of the twelve month period, and the untreated sample had 18% by weight of drug after the end of the 12 month period of storage at room temperature, then 2% by weight of the drug degraded in the untreated sample. Then preferably, the amount of degradation of the drug in the drug-resin complex treated with stabilizing agent after the 12 month period of storage at room temperature will be reduced to less than 2%, e.g., instead of 2% by weight of degradation, the treated drug preferably degrades by only about 1.75% by weight, more preferably only 1.5% by weight. Thus, a stabilized drug preferably has less than about 90% to 20%, preferably less than about 75% to 10%, and more preferably less than about 60% to 5%, of the degradation of an equivalent untreated drug.

Preferably, the stabilizing agent is included in the liquid carrier to associate it with drug-resin complexes. The stabilizing agent may be used in conjunction with coated drug resin complexes or non-coated drug resin complexes, as well as PEG-loaded complexes or those with no PEG-loading. With respect to coated complexes, the stabilizing agent may be used with coating material, including plasticizer or excluding plasticizer. In one embodiment, the stabilizing agent is not covalently bound to the drug resin complex. In another embodiment, the stabilizing agent is covalently bound to the drug resin complex.

Although any suitable drug can be included according to the invention that is capable of complexation to an ion exchange resin, and for which controlled release is desired, preferred drugs that can be included in the drug/resin complex include, but are not limited to, one or more analeptic agents; analgesics (e.g., aspirin, codeine, morphine, dihydromorphone, oxycodone, etc.); anesthetic agents; anti-asthmatics (e.g., theophylline); anti-arthritic agents; anti-cancer agents; anti-cholinergic agents; anti-convulsant agents (e.g., phenobarbital sodium, phenytoin sodium, valproate sodium barbiturates, amylobarbitone sodium, butabarbital sodium, secobarbital sodium); anti-epileptics (e.g., phenytoin, meprobamate and nitrezepam); anti-depressant agents; antidiabetics (e.g., insulin); anti-diarrheal agents; anti-helminthic agents; antihistamines (e.g., dimenhydrinate, diphenhydramine, chlorpheniramine and dexchlorpheniramine maleate); anti-hyperlipidemic agents; antihypertensives (e.g., clonidine, methyldopa; captopril); antihypotensives (e.g., propranolol, clonidine); anti-infective agents; anti-inflammatory agents; non-steroidal anti-inflammatory agents (e.g., naproxyn, diclofenac, indomethacin, ibuprofen, sulindac, meclofenamate sodium, tolmetin sodium); anti-emetics (e.g., metoclopramide); anti-migraine agents; anti-neoplastic agents; anti-Parkinsonism drugs; anti-pruritic agents; antipsychotic agents; anti-pyretic agents; anti-spasmodics (e.g., atropine, scopolamine); anti-tubercular agents; antibiotics (e.g., tetracyclines, such as doxycycline); antacids; antiulcer agents; anti-tussive agents and expectorants (e.g., codeine phosphate); anti-viral agents; anxiolytic agents; appetite suppressants (anorexic agents); attention deficit disorder and attention deficit hyperactivity disorder drugs; cardiovascular agents including calcium channel blockers, antianginal agents, central nervous system (“CNS”) agents, beta-blockers and antiarrhythmic agents; bronchodilators (e.g., albuterol); central nervous system stimulants; diuretics (e.g., ethacrynic acid, bendrofluazide); genetic materials; hormonolytics; hypnotics; hypercalcemics (e.g., etidronate disodium); hypoglycemic agents; immunosuppressive agents; antimuscarinics (e.g., glycopyrrolate); genitourinary smooth muscle relaxants; beta-agonists; narcotic antagonists; nicotine; nutritional agents; parasympatholytics; peptide drugs; antihemorrhoidals; psychostimulants; psychotropics; mucolytics; sedatives; decongestants; laxatives; vitamins (e.g., leucovorin calcium); sialagogues, steroids (e.g., hydrocortisone, triamcinolone, prednisone); smoking cessation agents; sympathomimetics; tranquilizers; vasodilators (e.g., nifedipine, papaverine, diltiazem and nicardirine); tocolytic agents; well as salts, hydrates, and solvates of the same.

The drug may be selected based on inclusion in the molecule of a functional group, such as an amino group, that will readily bind to a complexing agent, such as an ion-exchange resin. Any drug that bears an acidic or a basic functional group, for example, one or more of an amine, imine, imidazoyl, guanidine, pyridinyl, quaternary ammonium, or other basic group, or a carboxylic, phosphoric, phenolic, sulfuric, sulfonic or other acidic group, can be bound to a resin of the opposite charge. Exemplary drugs compatible with the present invention are generally described in, for example, WO 98/18610 by Van Lengerich, U.S. Pat. No. 7,070,806, U.S. Pat. No. 6,512,950, U.S. Pat. No. 5,202,128 and U.S. Pat. No. 4,996,047, the contents of each of which is incorporated herein by express reference thereto.

Suitable examples of the above families of drugs for use in the present invention may also include one or more of the following, across or within each family.

Alpha-adrenergic agonists that can be used include adrafinil, adrenolone, amidephrine, apraclonidine, budralazine, clonidine, cyclopentamine, detomidine, dimetofrine, dipivefrin, ephedrine, epinephrine, fenoxazoline, guanabenz, guanfacine, hydroxyamphetamine, ibopamine, indanazoline, isometheptene, mephentermine, metaraminol, methoxamine, methylhexaneamine, metizoline, midodrine, modafinil, moxonidine, naphazoline, norepinephrine, norfenefrine, octodrine, octopamine, oxymetazoline, phenylephrine hydrochloride, phenylpropanolamine hydrochloride, phenylpropyl-methylamine, pholedrine, propylhexedrine, pseudoephedrine, rilmenidine, synephrine, talipexole, tetrahydrozoline, tiamenidine, tramazoline, tuaminoheptane, tymazoline, tyramine, and xylometazoline.

Beta-adrenergic agonists that can be used include albuterol, bambuterol, bitolterol, carbuterol, clenbuterol, clorprenaline, denopamine, dioxethedrine, dopexamine, ephedrine, epinephrine, etafedrine, ethylnorepinephrine, fenoterol, formoterol, hexoprenaline, ibopamine, isoetharine, isoproterenol, mabuterol, metaproterenol, ethoxyphenamine, oxyfedrine, pirbuterol, prenalterol, procaterol, protokylol, reproterol, rimiterol, ritodrine, salmeterol, soterenol, terbutaline, tretoquinol, tulobuterol, and xamoterol.

Alpha-adrenergic blockers that can be used include amosulalol, rotinolol, dapiprazole, doxazosin, ergoloid mesylates, fenspiride, indoramin, labetalol, naftopidil, nicergoline, prazosin, tamsulosin, terazosin, tolazoline, trimazosin, and yohimbine.

Beta-adrenergic blockers that can be used include acebutolol, alprenolol, amosulalol, arotinolol, atenolol, befimolol, betaxolol, bevantolol, bisoprolol, bopindolol, bucumolol, bufetolol, bufuralol, bunitrolol, bupranolol, butidrine hydrochloride, butofilol, carazolol, carteolol, carvedilol, celiprolol, cetamolol, cloranolol, dilevalol, epanolol, esmolol, indenolol, labetalol, levobunolol, mepindolol, metripranolol, metoprolol, moprolol, nadolol, nadoxolol, nebivalol, nifenalol, nipradilol, oxprenolol, penbutolol, pindolol, practolol, pronethalol, propranolol, sotalol, sulfmalol, talinolol, tertatolol, tilisolol, timolol, toliprolol, and xibenolol.

Narcotic analgesics that can be used include alfentanil, benzylmorphine, codeine, codeine methyl bromide, codeine phosphate, codeine sulfate, desomorphine, dihydrocodeine, dihydrocodeinone enol acetate, dihydromorphine, ethylmorphine, hydrocodone, hydromorphone, methadone hydrochloride, morphine, morphine hydrochloride, morphine sulfate, nicomorphine, normethadone, normorphine, opium, oxycodone, oxymorphone, phenoperidine, and propiram.

Non-narcotic analgesics that can be used include aceclofenac, acetaminophen, acetanilide, acetylsalicylsalicylic acid, aspirin, carbamazepine, dihydroxyaluminum acetylsalicylate, fenoprofen, fluproquazone, ibufenac, indomethacin, ketorolac, magnesium acetylsalicylate, morpholine salicylate, naproxen, phenacetin, phenyl salicylate, salacetamide, salicin, salicylamide, sodium salicylate, and tolfenamic acid.

Anorexics that may be used include aminorex, amphecloral, amphetamine, benzphetamine, chlorphentermine, clobenzorex, cloforex, clortermine, cyclexedrine, dextroamphetamine sulfate, diethylpropion, diphemethoxidine, n-ethylamphetamine, fenbutrazate, fenfluramine, fenproporex, furfurylmethyl amphetamine, levophacetoperane, mazindol, mefenorex, metamfepramone, methamphetamine, norpseudoephedrine, pentorex, phendimetrazine, phenmetrazine, phentermine, phenylpropanolamine hydrochloride, picilorex, and sibutramine.

Antiallergics that may be used include amlexanox, astemizole, azelastine, cromolyn, fenpiprane, ibudilast, lodoxamide, nedocromil, oxatomide pemirolast, pentigetide, picumast, repirinast, suplatast tosylate, tazanolast, tranilast, and traxanox.

Antianginals that can be used include acebutolol, alprenolol, amiodarone, amlodipine, arotinolol, atenolol, barnidipine, bepridil, bevantolol, bucumolol, bufetolol, bufuralol, bunitrolol, bupranolol, carazolol, carteolol, celiprolol, cinepazet maleate, diltiazem, elgodipine, epanolol, felodipine, gallopamil, imolamine, indenolol, isosorbide dinitrate, sradipine, limaprost, mepindolol, metoprolol, molsidomine, nadolol, nicardipine, nicorandil, nifedipine, nifenalol, nilvadipine, nipradilol, nisoldipine, nitroglycerin, oxprenolol, oxyfedrine, ozagrel, penbutolol, pentaerythritol tetranitrate, pindolol, pronethalol, propranolol, ranolzazine, somotiadil, sotalol, terodiline, timolol, toliprolol, trolnitrate phosphate, verapimil, and zatebradine.

Antiasthmatics that can be used include amlexanox, azelastine, cromolyn, ibudilast, ketotifen, montelukast, nedocromil, oxatomide, pranlukast, seratrodast, suplatast tosylate, tiaramide, traxanox, zafirlukast, and zileuton.

Antibacterials or antibiotics can be used. The general classes of aminoglycosides, carbacephems, carbapenems, cephalosporins, cephamycins, penicillins, polypeptides, tetracyclines (e.g., doxycycline), etc. can be used. Specific antibacterials or antibiotics that can be used include amikacin, dihydrostreptomycin, kanamycin, neomycin, neomycin undecylenate, spectinomycin, streptomycin, loracarbef, biapenem, cefaclor, cefazolin, cefepime, cephalosporin C, cefbuperazone, andinocillin, amoxicillin, ampicillin, cloxacillin, metampicillin, penicillin G benzathine, penicillin G procaine, penicillin V, cloxacillin sodium, (di)cloxacillin sodium, nafcillin sodium, oxacillin sodium, carbenicillin indanyl sodium, novobiocin sodium, nitrofurantoin sodium, piperacillin, amphomycin, vancomycin, viomycin, apicycline, chlortetracycline, methacycline, doxycycline, and tetracycline.

Synthetic antibacterials such as quinolones and analogs, sulfonamides, etc. can be used. Specific synthetic antibacterials that can be used include cinoxacin, lomefloxacin, nalidixic acid, oxolinic acid, acetyl sulfa-methoxypyrazine, mafenide, succinylsulfathiazole, sulfacetamide, sulfadiazine, and sulfaloxic acid.

Anticholinergics that can be used include adiphenine hydrochloride, aminopentamide, atropine, chlorphenoxamine, cyclodrine, mecloxamine pentapiperide, phencarbamide, pridinol, and scopolamine.

Antidepressants that can be used include bicyclics, hydrazides, hydrazines, pyrrolidones, tetracyclics, tricyclics, etc. Specific antidepressants that can be used include binedaline, nefopam, trazodone, iproniazid, rolipram, maprotiline, adinazolam, amitriptyline, clomipramine, imipramine, nortriptyline, primipramine, adrafinil, milnacipran, nefazodone, and zimeldine.

Synthetic antifungals that can be used include allylamines, imidazoles, thiocarbamates, triazoles, etc. Specific synthetic antiflingals that can be used include butenafme, bifonazole, butoconazole, chlordantoin, clotrimazole, tolciclate, fluconazole, acrisorcin, exalamide, triacetin, and zinc propionate. Nonsteroidal anti-inflammatories that can be used include aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, etc. Specific nonsteroidal anti-inflammatories that can be used include flufenamic acid, terofenamate, acemetacin, clopirac indomethacin, metiazinic acid, fenbufen, clidanac, alminoprofen, bucloxic acid, ketoprofen, naproxen, tiaprofenic acid, difenamizole, apazone, mofebutazone, phenylbutazone, acetaminosalol, lysine acetylsalicylate, parsalmide, ampiroxicam, bendazac, nabumetone, superoxide dismutase, and zileuton.

Antispasmodics that can be used include alibendol, ambucetamide, aminopromazine, apoatropine, bevonium methyl sulfate, bietamiverine, butaverine, butropium bromide, caroverine, cimetropium bromide, cinnamedrine, clebopride, cyclonium iodide, difemerine, diisopromine, dioxaphetyl butyrate, diponium bromide, drofenine, emepronium bromide, fenalamide, fenoverine, flavoxate, flopropione, gluconic acid, hydramitrazine, hymecromone, octamylamine, pentapiperide, phloroglucinol, pinaverium bromide, piperilate, prifinium bromide, proxazole, racefimine, rociverine, spasmolytol, sultroponium, tigloidine, tiropramide, tricromyl, trimebutine, and xenytropium bromide.

Antiulceratives that can be used include acetoxolone, aldioxa, arbaprostil, benexate hydrochloride, carbenoxolone, cetraxate, cimetidine, colloidal bismuth subcitrate, ebrotidine, ecabet, enprostil, esaprazole, famotidine, gefarnate, guaiazulene, irsogladine, lansoprazole, misoprostol, nizatidine, omeprazole, ornoprostil, pantoprazole, pifarnine, pirenzepine, plaunotol, polaprezinc, rabeprazole, ranitidine, rebamipide, rioprostil, rosaprostol, rotraxate, roxatidine acetate, sofalcone, spizofurone, sucralfate, telenzepine, teprenone, trimoprostil, trithiozine, troxipide, and zolimidine.

Antivirals such as purines, pyrimidines, etc. can be used. Specific antivirals that can be used include acyclovir, cidofivir, cytarabine, dideoxyadenosine, didanosine, edoxudine, famciclovir, floxuridine, ganciclovir, idoxuridine, inosine pranobex, lamivudine, penciclovir, sorivudine, stavudine, zidovudine, acemannan, amantadine, amidinomycin, lysozyme, nevirapine, and ribavirin.

Anxiolytics such as arylpiperazines, benzodiazepine derivatives, carbamates, etc. can be used. Specific anxiolytics that can be used include buspirone, lesopitron, alprazolam, bromazepam, diazepam, fludiazepam, loxapine, metaclazepam, prazepam, cyclarbamate, meprobamate, abecarnil, benzoctamine, glutamic acid, mephenoxalone, and pazinaclone.

Calcium channel blockers such as arylalkylamines, dihydropyridine derivatives, piperazine derivatives, etc. can be used. Specific calcuim channel blockers that can be used include bepridil, diltiazem, gallopamil, terodiline, amlodipine, benidipine, lercanidipine, nicardipine, cinnarizine, and fantofarone.

Dopamine receptor agonists can be used. Specific dopamine receptors that can be used include bromocriptine, cabergoline, camioxirole, dopexamine, fenoldopam, ibopamine, lisuride, pergolide, pramipexole, quinagolide, ropinirole, roxindole, and talipexole. Dopamine receptor antagonists can be used. Specific dopamine receptor antagonists that can be used include amisulpride, clebopride, domperidone, metoclopramide, mosapramine, nemonapride, romoxipride, risperidone, sulpiride, sultopride, and ziprasidone.

Narcotic antagonists can be used. Specific narcotic anagonists that can be used include amiphenazole, cyclazocine, levallorphan, nalmefene, nalorphine, naloxone, and naltrexone.

Protease inhibitors can be used. Specific protease inhibitors that can be used include aprotinin, camostat, gabexate, nafamostat, and urinastatin.

Respiratory stimulants can be used. Specific respiratory stimulants that can be used include ahnitrine, bemegride, cropropamide, crotethamide, dimefline, dimorpholamine, doxapram, ethamivan, fominoben, lobeline, mepixanox, nikethamide, picrotoxin, pimeclone, pyridofylline, sodium succinate, and tacrine.

Retroviral protease inhibitors can be used. Specific retroviral protease inhibitors that can be used include indinavir, and ritonavir.

Reverse transcriptase inhibitors can be used. Specific reverse transcriptase inhibitors that can be used include delavirdine, didanosine, dideoxyadenosine, foscarnet sodium, lamivudine, nevirapine, stavudine, suramin sodium, zalcitabine, and zidovudine.

Sedatives such as benzodiazepine derivatives can be used. Specific sedatives that can be used include brotizolam, cinolazepam, doxefazepam, estazolam, flunitrazepam, flurazepam, haloxazolam, loprazolam, lormetazepam, nitrazepam, quazepam, temazepam, and triazolam.

Cerebral vasoldilators can be used. Specific cerebral vasodilators that can be used include bencyclane, cinnarizine, citicoline, cyclandelate, ciclonicate, eburnamonine, fasudil, fenoxedil, flunarizine, ibudilast, ifenprodil, lomerizine, nafronyl, nicametate, nicergoline, nimodipine, papaverine, pentifylline, tinofedrine, vincamine, vinpocetine, and viquidil.

Coronary vasodilator can be used. Specific coronary vasodilators that can be used include amotriphene, bendazol, benfurodil hemisuccinate, benziodarone, chloracizine, chromonar, clobenfurol, clonitrate, cloricromen, dilazep, dipyridamole, droprenilamine, efloxate, erytlirityl tetranitrate, etafenone, fendiline, floredil, ganglefene, heart muscle extract, hexobendine, itramin tosylate, khellin, lidoflazine, mannitol hexanitrate, medibazine, pentaerythritol tetranitrate, pentrinitrol, perhexiline, pimethylline, prenylamine, propatyl nitrate, pyridofylline, trapidil, tricromyl, trimetazidine, trolnitrate phosphate, and visnadine.

Peripheral vasodilator can be used. Specific peripheral vasodilators that can be used include bamethan, bencyclane, betahistine, bradykinin, brovincamine, bufeniode, buflomedil, butalamine, cetiedil, ciclonicate, cmepazide, cyclandelate, eledoisin, fenoxedil, flunarizine, hepronicate, ifenprodil, iloprost, inositol niacinate, isoxsuprine, kallidin, kallikrein, moxisylvyte, nafronyl, nicametate, nicergoline, nicofuranose, nicotinyl alcohol, nylidrin, pentifylline, pentoxifylline, piribedil, suloctidil, tolazoline, and xanthinol niacinate.

Antiamebics that can be used include arstiinol, bialamicol, carbarsone, cephaeline, chlorbetamide, chloroquine, chlorphenoxamide, chlortetracycline, dehydroemetine, dibromopropamidine, diloxanide, diphetarsone, emetine, fumagillin, glaucarubin, iodoquinol, paromomycin, phanquinone, polybenzarsol, propamidine, quinfamide, secnidazole, sulfarside, teclozan, tetracycline, thiocarbamizine, thiocarbarsone, and tinidazole. As noted above, it should be understood that any combination of any one or more of the above drug products (either within or between indications) may be complexed and coated according to the invention, preferably in association with a solid or liquid composition.

Preferably, some specific drugs that bear acidic or basic functional groups and thus may be complexed with an ion exchange resin include, but are not limited to, Acetylsalicylic acid, Alendronic acid, Alosetron, Amantadine, Amlopidine, Anagrelide, Argatroban, Atomoxetine, Atrovastatin, Azithromycin dihydrate, Balsalazide, Bromocriptan, Bupropion, Candesartan, Carboplatin, Ceftriaxone, Clavulonic acid, Clindamycin, Cimetadine, Dehydrocholic (acid), Dexmethylphenidate, Diclofenac, Dicyclomine, Diflunisal, Diltiazem, Donepezil, Doxorubicin, Doxepin, Doxycycline, Epirubicin, Etodolic acid, Ethacrynic acid, Felodipine, Fenoprofen, Fluoxetine, Furosemide, Gemfibrozil, Glipizide, Hydroxyzine, Ibuprofen, Imipramine, Levothyroxine, Maprolitline, Meclizine, Methadone, Methylphenidate, Metoclopramide, Minocycline, Mitoxantone, Moxifloxacin, Mycophenolic acid, Naproxen, Niflumic acid, Ofloxacin, Ondansetron, Oxycodone, Pantoprazole, Paroxetine, Pergolide, Pramipexole, Phenytoin, Pravastain, Prednisolone, Probenecid, Promethazine, Propranolol, Rabeprazole, Risedronic acid, Retinoic acid, Ropinirole, Selegiline, Sulindac, Tamsulosin, Telmisertan, Terbinafine, Theophyline, Tiludronic Acid, Tinzaparin, Ticarcillin, Valproic acid, Verapamil, Salicylic acid, Sevelamer, Ziprasidone, Zoledronic acid, Acetophenazine, Albuterol, Almotriptan, Amitriptyline, Amphetamine, Atracurium, Beclomethasone, Benztropine, Biperiden, Bosentan, Bromodiphenhydramine, Brompheniramine Carbinoxamine, Caffeine, Capecitabine, Carbergoline, Cetirizine, Chlocylizine, Chlorpheniramine, Chlorphenoxamine, Chlorpromazine, Citalopram, Clavunate potassium, Ciprofloxacin, Clemastine, Clomiphene, Clonidine, Clopidogrel, Codeine, Cyclizine, Cyclobenzaprine, Cyproheptadine, Delavirdine, Diethylpropion, Divalproex, Desipramine, Dexmethylphenidate, Dexbrompheniramine, Dexchlopheniramine, Dexchlor, Dextroamphetamine, Dexedrine, Dextromethorphan, Diphemanil methylsulphate, Diphenhydramine, Dolasetron, Doxylamine, Enoxaparin, Ergotamine, Ertepenem, Eprosartan, Escitalopram, Esomeprazole, Fenoldopam, Fentanyl, Fexofenadine, Fluvastatin, Fluphenazine, Fluticasone, Fosinopril, Frovatriptan, Gabapentin, Galatamine, Gatifloxacin, Gemcitabine, Haloperidol, Hyalurondate, Hydrocodone, Hydroxychloroquine, Hyoscyamine, Imatinib, Imipenem, Ipatropin, Lisinopril, Leuprolide, Levopropoxyphene, Losartan, Mesalamine, Mepenzolate, Meperidine, Mephentermine, Mesalimine, Mesoridazine, Metaproteranol, Metformin, Methdialazine, Methscopolamine, Methysergide, Metoprolol, Metronidazole, Mibefradil, Montelukast, Morphine, Mometasone, Naratriptan, Nelfinavir, Nortriptylene, Noscapine, Nylindrin, Orphenadrine, Oseltamivir, Oxybutynin, Papaverine, Pentazocine, Phendimetrazine, Phentermine, Pioglitazone, Pilocarpine, Prochloroperazine, Pyrilamine, Quetapine, Ranitidine, Rivastigmine, Rosiglitazone, Salmetrol, Sertaline, Sotalol, Sumatriptan, Tazobactam, Tacrolimus, Tamoxifen, Ticlopidine, Topiramate, Tolterodine, Triptorelin, Triplennamine, Triprolidine, Tramadol, Trovofloxacin, Ursodiol, Promazine, Propoxyphene, Propanolol, Pseudoephedrine, Pyrilamine, Quinidine, Oxybate sodium, Sermorelin, Tacrolimus, Tegaseroid, Teriparatide, Tolterodine, Triptorelin pamoate, Scoplolamine, Venlafaxine, Zamivir, Aminocaproic acid, Aminosalicylic acid, Hydromorphone, Isosuprine, Levorphanol, Melhalan, Nalidixic acid, and Para-aminosalicylic acid, and any combination thereof.

In more preferred embodiments, the drugs included in the drug component suitable for the composition of this invention include doxycycline, hydrocodone, oxybutynin, dextromethorphan, bupropion, venlafaxine, metoprolol, fluoxetine, or any salt thereof, or any combination thereof. Pharmaceutically acceptable salts of all of the above compounds may also be used.

Drug complexes are generally prepared by complexing each desired drug with one or more pharmaceutically acceptable ion-exchange resins. The complex is formed by reaction of a functional group of each drug with a functional group on one or more ion exchange resins. For example, a drug having a basic group such as an amino group can complex with an ion-exchange resin that bears an acidic group such as a sulfate or carboxylate group. Conversely, a drug that has in acidic group can complex with an ion-exchange resin that bears a basic group. The drug is released by being exchanging with appropriately charged ions within the gastrointestinal tract of a patient.

Ion-exchange resins are typically water-insoluble, cross-linked polymers containing covalently bound salt forming groups in repeating positions on the polymer chain. The ion-exchange resins suitable for use in these preparations consist of a pharmacologically inert organic or inorganic matrix. The organic matrix may be synthetic (e.g., polymers or copolymers of acrylic acid, methacrylic acid, sulfonated styrene, sulfonated divinylbenzene), or partially synthetic (e.g., modified cellulose and dextrans). The matrix can also be inorganic, e.g., silica gel, or aluminosilicates, natively charged or modified by the addition of ionic groups. The covalently bound salt forming groups may be strongly acidic (e.g., sulfonic or sulfate acid groups), weakly acidic (e.g., carboxylic acid), strongly basic (e.g., quaternary ammonium), weakly basic (e.g., primary amine), or a combination of acidic and basic groups. Other types of charged groups can also be used, including any organic group that bears an acidic or a basic functional group, for example, an amine, imine, imidazoyl, guanidine, pyridinyl, quaternary ammonium, or other basic group, or a carboxylic, phosphoric, phenolic, sulfuric, sulfonic or other acidic group.

In general, those types of ion-exchangers suitable for use in ion-exchange chromatography and for such applications as deionization of water are suitable for use in these controlled release drug preparations. Such ion-exchangers are described by H. F. Walton in “Principles of Ion Exchange” (pp. 312-343) and “Techniques and Applications of Ion-Exchange Chromatography” (pp. 344-361) in Chromatography. (E. Heftmann, editor), Van Nostrand Reinhold Company, New York (1975). The ion-exchange resins typically have exchange capacities below about 6 meq./g, and preferably below about 5.5 meq./g.

To complex the resin with an active pharmaceutical ingredient, the resin is charged and this charge is typically opposite to the charge on the selected drug. A few exemplary resins that can be used according to the invention include, but are not limited to, Dowex® resins and others made by Dow Chemical; Amberlite®, Amberlyst® and other resins made by Rohm and Haas; Indion® resins made by Ion Exchange, Ltd. (India), Diaion® resins by Mitsubishi; Type AG® and other resins by BioRad; Sephadex® and Sepharose® made by Amersham; resins by Lewatit, sold by Fluka; Toyopearl® resins by Toyo Soda; IONAC® and Whatman® resins sold by VWR; and BakerBond® resins sold by J T Baker. Particular resins known to be useful include Amberlite IRP-69® (Rohm and Haas), and INDION®, INDION 244®, and INDION 254® (Ion Exchange (India) Ltd.). These resins are generally sulfonated polymers composed of polystyrene cross-linked with divinylbenzene.

In preferred embodiments of the present invention, the resins are negatively charged and the drugs are positively charged. In other embodiments, as would be understood to one of ordinary skill in the art, positively charged resins, such as Duolite® AP143 (Rohm and Haas) and Polacrilin Potassium resins, including Amberlite® IRP-67 and Amberlite® FPA, may be used in conjunction with negatively charged drugs and any other pharmaceutically acceptable suitable excipients. Regardless of the charge pairing, the drug/resin forms are then coated according to the invention.

The size of the ion-exchange particles should be less than about 2 millimeters down to 0.001 μm, preferably from about 1000 μm to 0.1 μm, more preferably from about 500 μm to 1 μm, and most preferably about 150 μm to 45 μm. Commercially available ion-exchange resins (including Amberlite® IRP-69, INDION 244®, and INDION 254® and numerous other products) are typically available in several particle size ranges, and many have an available particle size range less than 150 μm.

Adsorption of each drug onto the resin particles, i.e., ion exchange resin particles to form the drug resin complex, is a well known technique as shown in U.S. Pat. No. 2,990,332 to Keating and U.S. Pat. No. 4,221,778 to Raghunathan. In general, the drug is mixed with an aqueous suspension of the resin, and the complex is then washed and dried. Adsorption of drug onto the resin may be detected by a change in the pH of the reaction medium or by a reduction in the concentration of dissolved drug in the reaction solvent. Generally, the pKₐ and the functional group(s) of, the drug help determine the type of resin that can be used.

A drug-resin complex may be formed by the reaction of a resin with a drug using standard techniques. For example, a sodium salt of a resin may be reacted with a cationic drug. The amounts of drug and resin necessary to form an effective drug resin complex will vary greatly. Among the factors to be considered in determining the ratio of drug to resin are the particular drug, the resin used, the reaction conditions, and the final dosage form required. The resin preferably has a high loading capacity for the drug in question. A small loading capacity may make the resulting dosage form overly bulky or expensive to produce. Actual loading of the drug on the resin particles can range from about 1 to 90 percent by weight, preferably 5 to 70 percent by weight, and more preferably 10 to 40 percent by weight, based on the weight of the drug-containing complex.

Binding of drug to resin can be accomplished according to four general reactions. In the case of a basic drug, these are: (a) resin (Na-form) plus drug (salt form); (b) resin (Na-form) plus drug (as free base); (c) resin (H-form) plus drug (salt form); and (d) resin (H-form) plus drug (as free base). All of these reactions except (d) have cationic by-products and these by-products, by competing with the cationic drug for binding sites on the resin, reduce the amount of drug bound at equilibrium. For basic drugs, stoichiometric binding of drug to resin is accomplished only through reaction (d).

Four analogous binding reactions can be carried out for binding an acidic drug to an anion exchange resin. These are: (a) resin (Cl-form) plus drug (salt form); (b) resin (Cl-form) plus drug (as free acid); (c) resin (as free base) plus drug (salt form); and (d) resin (as free base) plus drug (as free acid). All of these reactions except (d) have ionic by-products, and the anions generated when the reactions occur compete with the anionic drug for binding sites on the resin with the result that reduced levels of drug tend to be bound at equilibrium. For acidic drugs, stoichiometric binding of drug to resin is typically accomplished only through reaction (d).

In a preferred embodiment, production of the coated, drug resin complex includes at least the following steps: complexing a plurality of dry ion exchange resin forms with a drug component including at least one active pharmaceutical ingredient, in one embodiment suspending drug/resin complex in polyethylene glycol in water to associate polyethylene glycol with the ion exchange resin forms and then drying to remove excess water from the drug resin complex impregnated with polyethylene glycol, then coating the drug resin complex with Eudragit® NE 30D, and optionally, but preferably, suspending the coated drug resin complex in association with a liquid carrier that is present in an amount sufficient to form a suspension of the coated ion exchange resin forms.

Preparation of the drug/resin complex may include combining a plurality of ion exchange resin forms to sufficiently mix with a pharmaceutically effective amount of suitable active pharmaceutical ingredient. The ion exchange resins and drug/resin complex can be prepared by any suitable methods and/or materials available to those of ordinary skill in the art to provide a controlled release form of a desired drug component of one or more active pharmaceutical ingredients. Numerous pharmaceutically acceptable resins, and the process for complexing resins to drugs, are available commercially or can be made according to details available in the art, including, e.g., in U.S. Pat. Nos. 2,990,332, 4,221,778, and 4,847,077, European Patent No. 368682, and Great Britain Patent Nos. 824337 and 1218102, each of which is hereby incorporated herein by express reference thereto.

Additional details on preparing drug/resin complexes, particularly in connection with the PEG-loading aspect of the invention, are further described. Preferably an aqueous solution, such as water, is used. The impregnated drug/resin complex is preferably dried and then preferably sieved or otherwise arranged to a uniform size or range of sizes. PEG dissolved in an aqueous solution, such as water, can be added to the drug resin complex in one aspect of the invention. The PEG-loaded drug/resin complex is also preferably dried. Coating formulation, optional but preferred, is prepared, as is understood by one of ordinary skill in the art, and applied, such as through spraying, over and on the drug/resin complexes. Coated beads are preferably cured under suitable conditions, typically involving a temperature increased above room temperature. An anti-sticking agent can be added as necessary before the curing.

A stabilizing agent may be included at any point in the preparation of the coated drug-resin complexes. For example, a stabilizing agent may be added during either the complexation process or the coating process, or both. The stabilizing agent is optionally but preferably added after the drug-resin complex is formed. The drug-resin complex may be dried before or after adding the stabilizing agent. In a preferred embodiment, since neither resins nor drug-resin complexes are typically highly soluble in water, processes adding stabilizing agent are preferably carried out with the resin already suspended, such as in a liquid carrier. The stabilizing agent may be added to a liquid in which the resin is suspended or to one or more solid excipients, either before or after addition of the coated or uncoated drug/resin complexes. Any suitable stabilizing agent may be included. Preferably EDTA, citric acid, ascorbic acid, BHA, BHT, or a salt of any of the foregoing, or any combination thereof, are included in the stabilizing agent. More preferably, one or more of citric acid, ascorbic acid, BHA, BHT, or a salt thereof, are included in the stabilizing agent. An exemplary stabilizing agent includes or is citric buffer. The stabilizing agent may be present in any suitable amount, preferably from about 0.01 w/v percent to 2.5 w/v percent, and more preferably from about 0.05 w/v percent to 1 w/v percent, of the composition. An exemplary amount includes from about 0.1 w/v percent to 0.5 w/v percent, of the composition.

Advantageously, an antioxidant component including one or more antioxidants or materials providing an antioxidant effect can be included in an amount sufficient to increase the stability of the compositions of the invention. When included, the antioxidant component is preferably present in an amount of about 0.1 w/v percent to 2.5 w/v percent, more preferably about 0.2 w/v percent to 1 w/v percent. Preferably, a chelating agent and an antioxidant are included in liquid formulations of the invention to increase the stability of the coated drug/resin complex forms. In one preferred embodiment, the invention may include a citric buffer, ascorbic acid, BHT, BHA or a combination thereof, in an amount sufficient to increase the stability of the drug in the coated forms, preferably synergistically.

The pharmaceutical composition is suitable for any route of administration, including oral, topical, rectal, vaginal, nasal, or ophthalmic administration. Preferred embodiments of the invention are suitable for oral administration. Suitable orally administrable pharmaceutical compositions include forms such as a tablet, capsule, powder, sachet, suspension, syrup. More preferred forms include liquid formulations including the coated drug/resin complexes according to the invention. For aspects of the invention where a solid dosage form are preferred, however, the coated drug/resin complexes can be prepared in any suitable solid form available to those of ordinary skill in the art including tablets, capsules, caplets, powders, sachets, sprinkle granules, granules, pellets, or the like. In such solid forms, it should be understood that a solid carrier would alternatively be used in place of some or all the liquid carrier components described herein so as to provide a solid formulation. For example, solid drug/resin complex forms may be coated according to the invention so that the coating is sufficient to minimize bitterness or other undesirable characteristics of certain uncoated drug/resin complexes.

The preferred embodiment when a liquid carrier is included is a suspension. The liquid carrier preferably includes a suspending agent, a thickening agent, a preservative agent, a sweetening agent, a texture/mouth-feel agent in buffer solution to adjust the pH, and one or more other excipients generally known in the art, such as a wetting agent and a dispersing agent, if required to provide a stable suspension.

PEG may be included in the liquid carrier to create equilibrium or otherwise modify the release of PEG from the drug/resin complexes. When PEG is included, any suitable amount may be used to minimize or prevent diffusion or other release of PEG from the coated drug/resin complexes dispersed therein. Preferably, the amount of PEG included, when used, is from about 0.1 weight percent to 15 weight percent. In one embodiment, the amount of PEG included is substantially equivalent to the amount of PEG, on a weight percent basis, included in the drug/resin complexes.

A thickening agent or viscosity-enhancing agent may be included to generally improve the mouth-feel of the composition and/or to help coat the lining of the gastrointestinal tract. While any suitable thickening agent available to those of ordinary skill in the art may be included in the compositions of the present invention, a preferred thickening agent, when used, includes one or more of acacia, alginic acid bentonite, carbomers, carboxymethylcellulose calcium or sodium, cetostearyl alcohol, methyl cellulose, ethylcellulose, glycerin, gelatin guar gum, hydroxyethyl cellulose, hydroxymethyl cellulose, hydroxypropyl cellulose, hydroxypropyl methyl cellulose (“HPMC”), any other suitable cellulose-based component, maltodextrin, polyvinyl alcohol, povidone, propylene carbonate, propylene glycol alginate, sodium alginate, starch, sodium starch glycolate, starch tragacanth, and xanthan gum, or a combination thereof.

Any suitable sweetening agent available to those of ordinary skill in the art may be used according to the invention. Typically, the sweetening agent includes sorbitol, saccharin, acesulfame, e.g., acesulfame potassium, sucralose, xylitol, maltitol, sucrose, aspartame, fructose, neotame, sodium saccharate, glycyrrhizin dipotassium, mannitol, propylene glycol, invert sugar, and any combination thereof, or components containing a sweetening agent, such as one or more sucralose-containing components or saccharin-containing components, may be added to modify the taste of the composition. Alternatively, or in addition, a viscous sweetener such as one or more of a sorbitol solution, a syrup (sucrose solution) glycerin, liquid sugar or high-fructose corn syrup can be used and, in addition to sweetening effects, may also be useful to increase viscosity and to retard sedimentation. Alternatively, the carrier associated with the coated drug/resin complex may be at least substantially, or even entirely, free of natural sugar, or even of a sweetening agent.

A suitable buffering agent, as understood in the art, is used to adjust the pH of the liquid carrier. An exemplary buffering agent includes one or more of potassium phosphate dibasic, calcium carbonate, sodium bicarbonate, sodium and potassium hydroxide, citric acid, ascorbic acid, any combination thereof. Preferably, the buffering agent includes citric acid, ascorbic acid, or a combination thereof.

When included in the liquid carrier, any suitable preservative agent available to those of ordinary skill in the art may be included, typically in an amount sufficient to extend the shelf life or storage stability, or both, of at least the liquid carrier components. Preferred examples of a suitable preservative agent, when used, include sodium benzoate, butylated hydroxy toluene, butylated hydroxyanisole, ethylenediamine tetraacetic acid, paraoxybenzoic acid esters, methyl, ethyl, butyl, and propyl parabens, potassium sorbate, sodium metabisulfite, chlorobutanol, benzyl alcohol, phenylethylalcohol, dehydroacetic acid, sorbic acid, benzalkonium chloride (BKC), benzethonium chloride, phenol, phenylmercuric nitrate, thimerosal, and any combination thereof. A preservative agent may be added to the compositions at levels safe for ingestion, e.g., to improve storage stability. Such a preservative agent, if present, may be present in a conventional amount.

Although not required, any suitable stable flavoring agent available to those of ordinary skill in the art may be included in the present liquid carrier, typically to enhance patient compliance by making the compositions of the present invention more palatable. Such a flavoring agent, if present, may be present in a conventional amount.

Although not required, a colorant agent, when included, may be provided in an amount sufficient to provide the compositions with a more aesthetic and/or distinctive appearance and even to mask the appearance of the drug/resin complex. Any suitable colorant agent including one or more coloring materials available to those of ordinary skill in the art may be selected in any conventional amount.

Any suitable suspending agent available to those of ordinary skill in the art may be used according to the invention. A preferred suspending agent of the present invention are methylcellulose, sodium carboxymethyl cellulose and hydroxypropyl methyl cellulose (HPMC), polyvinylpyrrolidone, alginates, chitosan, dextrans, gelatin, polyethylene glycols, xanthan gum, microcrystalline cellulose, carboxymethylcellulose, Avicel® (manufactured by FMC Polymer Pa., USA), Carbopol® (manufactured by Noveon of Ohio, USA), liquid sugar, and HFCS 90, as well as polyoxyethylene- and polyoxypropylene ethers. Such a suspending agent, if present, may be present in a conventional amount.

A wetting agent (or “surfactant”) may be included in the liquid compositions of the present invention, although typically such wetting agents are present to facilitate impregnation of PEG into the drug/resin complexes. When used, the wetting agent may include any suitable type available to those of ordinary skill in the art. For example, the wetting agent may include quaternary ammonium compounds, such as benzalkonium chloride, benzethonium chloride and cetylpyridinium chloride; TPGS (i.e., d-alpha tocopheryl polyethylene glycol 1000 succinate), dioctyl sodium sulfosuccinate; polyoxyethylene alkylphenyl ethers, such as nonoxynol 9, nonoxynol 10, and octoxynol 9; poloxamers (polyoxyethylene and polyoxypropylene block copolymers); polyoxyethylene fatty acid glycerides and oils, such as polyoxyethylene (8) caprylic/capric mono- and diglycerides (e.g., Labrasol®, Gattefosse), polyoxyethylene (35) castor oil and polyoxyethylene (40) hydrogenated castor oil; polyoxyethylene alkyl ethers, such as polyoxyethylene (20) cetostearyl ether; polyoxyethylene fatty acid esters, such as polyoxyethylene (40) stearate; polyoxyethylene sorbitan esters, such as polysorbate 20 and polysorbate 80 (e.g., Tween® 80, ICI); propylene glycol fatty acid esters, such as propylene glycol laureate (e.g., Lauroglycol®, Gattefosse); sodium lauryl sulfate; fatty acids and salts thereof, such as oleic acid, sodium oleate and triethanolamine oleate; glyceryl fatty acid esters, for example glyceryl monostearate; sorbitan esters, such as sorbitan monolaurate, sorbitan monooleate, sorbitan monopalmitate and sorbitan monostearate; tyloxapol; lecithin; stearyl triethanolamine; laurylaminopropionic acid; and any combination thereof. Such a wetting agent, if present, may be used in a conventional amount, based on the total volume of the pharmaceutically acceptable carrier.

Preferably, the delay in release, or sustained release, occurs over greater than four hours to seven days, and more preferably greater than six hours to five days, for the coated complexes of the present invention. In more preferred exemplary embodiment, the drug release occurs over about eight hours, twelve hours, sixteen hours, twenty-four hours, or forty-eight hours. Further, the coatings of the invention are substantially free of any interaction with the drug component, particularly anionic drug components.

The term “about,” as used herein, should generally be understood to refer to both numbers in a range of numerals. Moreover, all numerical ranges herein should be understood to include each whole integer within the range. All percentages discussed herein should be understood to refer to weight percentages unless otherwise noted.

### EXAMPLES

The invention is further defined by reference to the following illustrative examples that may be used to prepare or administer the compositions of the present invention. These examples are for illustrative purposes only, and are not to be construed as limiting the appended claims.

### Example 1a

### Preparation of Dextromethorphan Polistirex

An amount of 400 g of Amberlite® IRP69 of sodium form was sieved to a desired size, 175 g dextromethorphan HBr and 4000 ml of water were added to a beaker and stirred mechanically for 6 hours. The mixture was filtered through a Busch funnel. The filtration cake was washed three times with excess amount of water in order to remove drugs that were not bound to the resin. The filtered cake was then fluid-bed dried for 1.5 hours in a Uni-Glatt fluid processor with the inlet temperature ranging from 40 to 70° C. The assay of final drug resin complex was 30% (w/w) dextromethorphan HBr in the drug/resin complex.

### Example 1b

### Preparation of a Oxybutynin Polistirex

An amount of 740 g of Amberlite® IRP69 of sodium form was sieved to a desired size, 222 g Oxybutynin HCl and 4000 ml of water were added to a beaker and stirred mechanically for 6 hours. The mixture was filtered through a Busch funnel. The filtration cake was washed three times with excess amount of water in order to remove drugs that were not bound to the resin. The filtered cake was air-dried. The assay of final drug resin complex was 23% (w/w) oxybutynin HCl in the drug/resin complex.

### Example 1c

### Preparation of Hydrocodone Polistirex

An amount of 400 g of Amberlite® IRP69 of sodium was sieved to a desired size, 150 g Hydrocodone bitartrate and 4000 ml of water were added to a beaker and stirred mechanically for 6 hours. The mixture was filtered through a Busch funnel. The filtration cake was washed three times with excess amount of water in order to remove drugs that were not bound to the resin. The filtered cake was then oven-dried at 40° C. The assay of final drug resin complex was 25% (w/w) hydrocodone bitartrate in the drug/resin complex.

### Example 1d

### Preparation of Venlafaxine Polistirex

An amount of 200 g of Amberlite® IRP69 of sodium form was sieved to a desired size, 95 g Venlafaxine HCl and 1500 ml of water were added to a beaker and stirred mechanically for 8 hours. The mixture was filtered through a Busch funnel. The filtration cake was washed three times with excess amount of water in order to remove drugs that were not bound to the resin. The filtered cake was then air-dried. The assay of final drug resin complex was 31% (w/w) Venlafaxine HCl in the drug/resin complex.

### Example 1e

### Preparation of Metoprolol Polistirex

An amount of 200 g of Amberlite® IRP69 of sodium form was sieved to a desired size, 95.1 g Metoprolol succinate and 1600 ml of water were added to a beaker and stirred mechanically for 8 hours. The mixture was filtered through a Busch funnel. The filtration cake was washed three times with excess amount of water in order to remove drugs that were not bound to the resin. The filtered cake was then air-dried. The assay of final drug resin complex was 30% (w/w) Metoprolol succinate in the drug/resin complex.

### Example 2

### Polyethylene Glycol Impregnation of Drug Resin Complex

Different amounts of PEG 3350 at 7 and 14 weight percent to the dried drug-resin complex, were dissolved in water at about 80 to 90 weight percent to the dry beads. Then, dried drug-resin complexes, such as those prepared according to Example 1, were added to each solution and mixed well. The drying process was carried out by either oven drying at 60° C. or fluid-bed drying in the Uni-Glatt fluid processor. The dried material is then passed through a Mesh 60 screen before use.

### Example 2a

### Polyethylene Glycol Treatment of Dextromathorphan Polistirex

To make 7% PEG to Dextromathorphan polistirex, 21 g of polyethylene glycol 3350 was dissolved in 270 ml water, then 300 g of dried Dextromathorphan polistirex, such as those prepared according to Example 1a, was added and mixed well. The wet cake was fluid-bed dried for 2 hours in a Uni-Glatt fluid processor with the inlet temperature ranging from 40 to 60° C. The dried material is then passed through a Mesh 60 screen before use.

### Example 2b

### Polyethylene Glycol Treatment of Oxybutynin Polistirex

To make 7% PEG to Oxybutynin polistirex, 22.4 g of polyethylene glycol 3350 was dissolved in 256 ml water, then 320 g of dried Oxybutynin polistirex, such as those prepared according to Example 1b, was added and mixed well. The drying process was carried out by oven drying at 60° C. The dried material is then passed through a Mesh 60 screen before use. To make 14% PEG to Oxybutynin polistirex, 44.8 g of polyethylene glycol 3350 was dissolved in 256 ml water, then 320 g of dried Oxybutynin polistirex, such as those prepared according to Example 1b, was added and mixed well. The drying process was carried out by oven drying at 60° C. The dried material is then passed through a Mesh 60 screen before use.

### Example 2c

### Polyethylene Glycol Treatment of Hydrocodone Polistirex

To make 7% PEG to Hydrocodone polistirex, 23.8 g of polyethylene glycol 3350 was dissolved in 330 ml water, then 340 g of dried Hydrocodone polistirex, such as those prepared according to Example 1c, was added and mixed well. The drying process was carried out by oven drying at 60° C. The dried material is then passed through a Mesh 60 screen before use. To make 14% PEG to Hydrocodone polistirex, 47.6 g of polyethylene glycol 3350 was dissolved in 330 ml water, then 340 g of dried Hydrocodone polistirex, such as those prepared according to Example 1c, was added and mixed well. The drying process was carried out by oven drying at 60° C. The dried material is then passed through a Mesh 60 screen before use.

### Example 2d

### Polyethylene Glycol Treatment of Venlafaxine Polistirex

To make 8% PEG to Venlafaxine polistirex: 22 g of polyethylene glycol 3350 was dissolved in 210 ml water, then 280 g of dried Venlafaxine polistirex, such as those prepared according to Example 1e, was added and mixed well. The drying process was carried out by oven drying at 60° C. The dried material is then passed through a Mesh 60 screen before use.

### Example 2e

### Polyethylene Glycol Treatment of Metoprolol Polistirex

To make 8% PEG to Metoprolol polistirex, 21 g of polyethylene glycol 3350 was dissolved in 210 ml water, then 270 g of dried Metoprolol polistirex, such as those prepared according to Example 1f, was added and mixed well. The drying process was carried out by oven drying at 60° C. The dried material is then passed through a Mesh 60 screen before use.

### Example 3a

### Preparation of Eudragit® NE Coating Formulation According to the Invention

A typical procedure for preparing aqueous polymeric suspension was as follows: 100 ml of water was heated to 70 to 80° C. before 30 g GMS and talc as well as 1.3 g of simethicone emulsion 30% were added. The mixture was homogenized for 10 min at 4500 rpm by a Silverson L4RT homogenizer then cooled down to room temperature under gentle stirring. An amount of 320 g Eudragit® NE-30D was added to the suspension under stirring. Additional water was added to make the total weight equal to 610 g to 630 g. The mixture was filtered through a Mesh 40 screen before being used for coating.

### Example 3b

### Preparation of Eudragit® RS/RL Coating Formulation

A typical procedure for preparing aqueous polymeric suspension was as follows: 100 ml of water was heated to 70 to 80° C. before 5 g GMS (anti-sticking agent), 13.8 g dibutyl sebacate (DBS, a plasticizer) and 1.3 g simethicone emulsion 30% were added to the solution. The mixture was homogenized for 10 minutes at 4500 rpm then cooled down to room temperature under gentle stirring. An amount of 213 g Eudragit® RS-30D and 17 g Eudragit® RL-30D (RS to RL ratio being 93 to 7) was added to the GMS/DBS suspension under stirring. Additional water was added to make the total weight equal to 620 g. The mixture was filtered through a screen to discard big particles before being used for coating. Another anti-sticking agent, such as talc, was optionally used in some preparations.

### Example 4

### Comparative Preparation of Coated Resin-Drug Complexes

About 320 g of drug resin complex (cores) was transferred to the product chamber of a Uni-Glatt processor. Coating formulation prepared according to Examples 3a and 3b was sprayed onto the fluidized complex particles at an inlet temperature range of 20 to 40° C. for Eudragit® NE 30D and 30 to 60° C. for RL-30D RS and RL for Eudragit® NE 30D. The coating level was determined by the weight change of the coating formulation used. The coated beads were passed through a screen to discard big particles and polymer lumps and to obtain a desired size. Dextromethorphan polistirex was coated by either Eudragit® NE 30D or Eudragit® RS and RL 30D.

### Example 5

### Curing of Aqueous Coated Beads According to the Invention

About 200 g of coated beads prepared according to Example 4 were mixed with 12 g of talc before placing the beads in an oven at 40° C. for 24 hours to cure each type of coated beads including Eudragit® NE, RS, and RL.

### Example 6

### Preparation of Suspension Dosage Forms for Coated Dextromethorphan Polistirex

An exemplary preparation procedure for dextromethorphan polistirex suspension base (citric acid concentration around 0.15% w/v, i.e., suspension I, as described below in Example 10) included first mixing 3.4 g of citric acid, 650 g of xylitol, 34.2 g Avicel® RC 591, and 757.2 g glycerin in 1800 g of H₂O. An amount of 57.49 g of the preservative/propylene glycol (PG) mixture, which contains 51.33 g polylene glycol, 5.13 g methyl paraben, 1.03 g propyl paraben, and 6.87 g xanthan gum, was added. Then an amount of 50 g of color and flavor aqueous solution was added to obtain the suspension base (i.e., vehicle).

The dextromethorphan suspension base with citric acid concentration of 0.5% w/v (i.e., suspension II, as described below in Example 10) was prepared simultaneously according to the same procedure except using the combination of 3.3 g citric acid and 1.4 g sodium citrate dihydrate per 1000 ml suspension base.

The dextromethorphan suspensions were prepared by adding 68.7 g of aqueous Eudragit® NE-coated dextromethorphan polistirex (10% coating, 7% PEG-treated, Drug Loading=26.2%) into the 3000 ml of well-mixed vehicles to make dextromethorphan be 30 mg per 5 ml of suspension. The pH of the suspension was measured and adjusted to 3.8 with addition of sodium citrate. The specific gravity of the final suspension is about 1.13 g/ml.

### Example 7

### Preparation of Suspension Dosage Forms for Coated Oxybutynin Polistirex

An exemplary preparation procedure for the oxybutynin polistirex vehicle was as follows. An amount of 0.34 g of citric acid, 12.5 g of starch, 3.0 g of PEG 3350, 200.0 g of liquid sugar, 150.0 g of HFCS 90 in 500 ml of H₂O were mixed. Then, 9.8 g of the preservative solution, which contains 8.0 g PG, 1.5 g methyl paraben, and 0.3 g propyl paraben, were added. Finally, 4.0 g of xanthan gum and suitable quantities of colors and flavors were added to obtain the oxybutynin vehicle.

The oxybutynin polistirex suspensions were prepared by adding certain amounts of aqueous Eudragit® NE-coated oxybutynin polistirex into the well-mixed vehicles. For oxybutynin polistirex suspension containing non-PEG-treated aqueous Eudragit® NE-coated oxybutynin polistirex, 1.56 g of Eudragit® NE-coated oxybutynin polistirex (15% coating, drug loading=19.2%) per 100 ml suspension base were used to make oxybutynin in the amount of 15 mg per 5 ml of suspension.

For oxybutynin polistirex suspension containing 14% PEG-treated aqueous Eudragit® NE-coated oxybutynin polistirex, 1.85 g of Eudragit® NE-coated oxybutynin polistirex (25% coating, drug loading=16.2%) per 100 ml suspension base were used to make oxybutynin in an amount of 15 mg per 5 ml of suspension. The pH of each suspension was measured and adjusted to between 3.5 and 4.5.

### Example 8

### Preparation of Suspension Dosage Forms for Coated Hydrocodone Polistirex and Un-Coated Chlorpheniramine Polistirex

An exemplary preparation procedure for the a coated hydrocodone polistirex and uncoated chlorpheniramine polistirex vehicle included mixing 2.0 g of ascorbic acid, 50.0 g of starch, 12.0 g of PEG 3350, 800.0 g of liquid sugar, 600.0 g of HFCS 90 in 2000 ml of H₂O. Then, 39.4 g of the preservative solution, which contains 32.2 g PG, 6.0 g methyl paraben, and 1.2 g propyl paraben, was added. Finally, 16.0 g of xanthan gum and suitable quantities of colors and flavors were added to obtain the coated hydrocodone polistirex and uncoated chlorpheniramine polistirex vehicle.

These coated hydrocodone polistirex and uncoated chlorpheniramine polistirex suspensions were prepared by adding certain amounts of aqueous Eudragit® NE-coated hydrocodone polistirex and chlorpheniramine-complex beads into the well-mixed vehicles. For coated hydrocodone polistirex and uncoated chlorpheniramine polistirex suspensions containing non-PEG-treated aqueous Eudragit® NE-coated hydrocodone polistirex, 1.31 g of Eudragit® NE-coated hydrocodone polistirex (25% coating, drug loading=15.27%) and 0.48 g of chlorpheniramine-complex beads (drug loading=33.3%) per 100 ml suspension base were used. For coated hydrocodone polistirex and uncoated chlorpheniramine polistirex suspensions containing 14% PEG-treated aqueous Eudragit® NE-coated hydrocodone polistirex, 1.67 g of Eudragit® NE-coated hydrocodone polistirex (20% coating, drug loading=12.0%) and 0.48 g of chlorpheniramine-complex beads (drug loading=33.3%) per 100 ml suspension base were used. Each 5 ml of the suspension contains hydrocodone polistirex equivalent to 10 mg of hydrocodone bitartrate and chlorpheniramine polistirex equivalent to 8 mg of chlorpheniramine maleate. The pH of the suspension was measured and adjusted to around 4.78.

### Example 9

### Dissolution Data

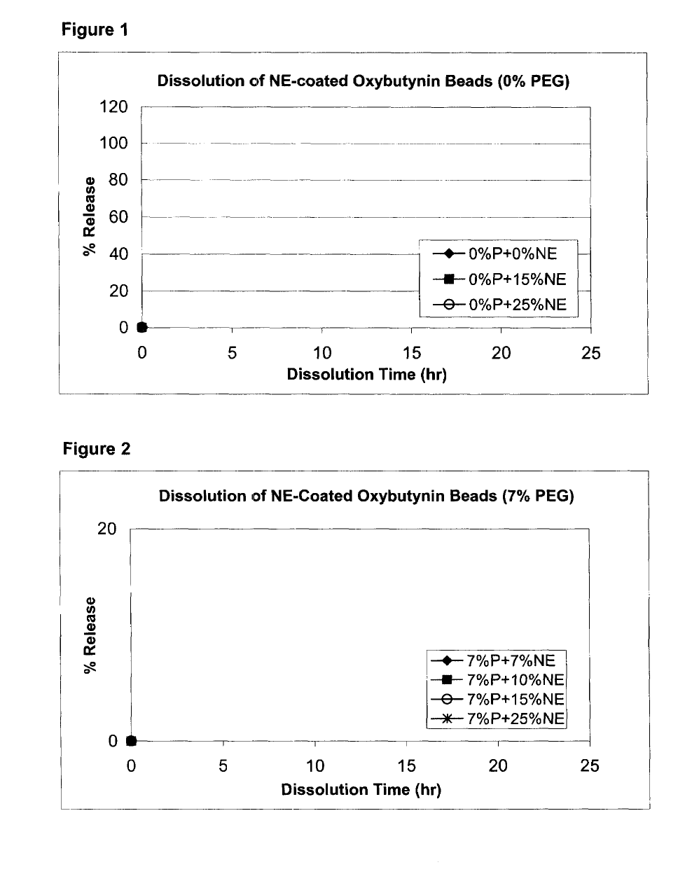

FIGS. 1-3 graphically represent the effects of varying amounts of PEG and Eudragit® NE 30D on the in vitro dissolution of coated Oxybutynin resin complexes, when measured using a 900 ml of dissolution medium (0.1N HCl and 0.4N NaCl), a USP type II paddle at 75 rpm for oxybutynin polistirex (50 rpm for most other drug complex resins) positioned around 1.5 cm above the bottom of the vessel and at 37±0.5° C., using HPLC with DAD detector at different wavelengths for various drugs listed.

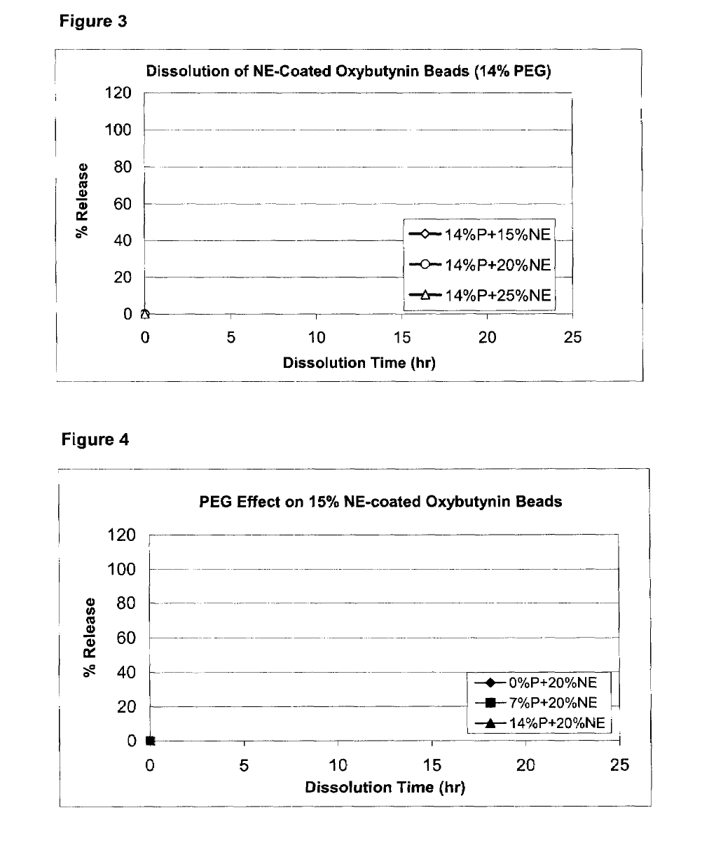

In FIG. 1, the PEG was controlled at a baseline level. In FIG. 2, the percentage of PEG was increased to 7%. The addition of PEG increased the cumulative percentage of Oxybutynin released. In FIG. 3, PEG levels were increased to 14%, and there was a significant increase in the extent of drug released at the end of dissolution. FIG. 4 illustrates the effect of PEG changes on constant amounts of Eudragit® NE 30D coating. As PEG was increased to 14%, the percent cumulative release of the drug approximated 100%, in contrast to when no PEG is present, wherein the cumulative release at 24 hours was approximately 50%.

The results indicated that the dissolution profiles of oxybutynin were surprisingly and unexpectedly regulated via Eudragit® NE 30D coating levels, with or without PEG treatment. Use of PEG surprisingly increased the extent of drug released at the end of the 24-hour dissolution period. Dissolution studies of the non-PEG treated, Eudragit® NE 30D coated Oxybutynin resin complex clearly showed that the Eudragit® NE 30D coating significantly decreased the drug release rate at levels of, e.g., 15% coating. The non-PEG treated, Eudragit® NE 30D coated Oxybutynin resin complex had the least cumulative release of drug over time compared to PEG treated, Eudragit® NE 30D coated Oxybutynin resin complexes. Oxybutynin release was more complete with 7% of PEG treated drug resin complex than non-PEG treated drug resin complexes, while a higher percentage of drug release surprisingly occurred with 14% PEG, Eudragit® NE 30D coated drug resin complexes.

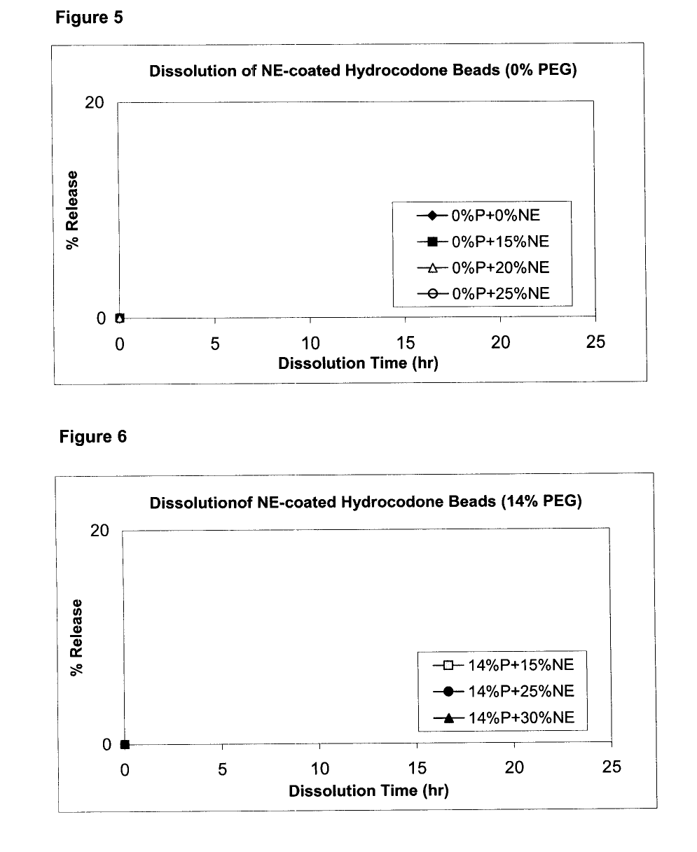

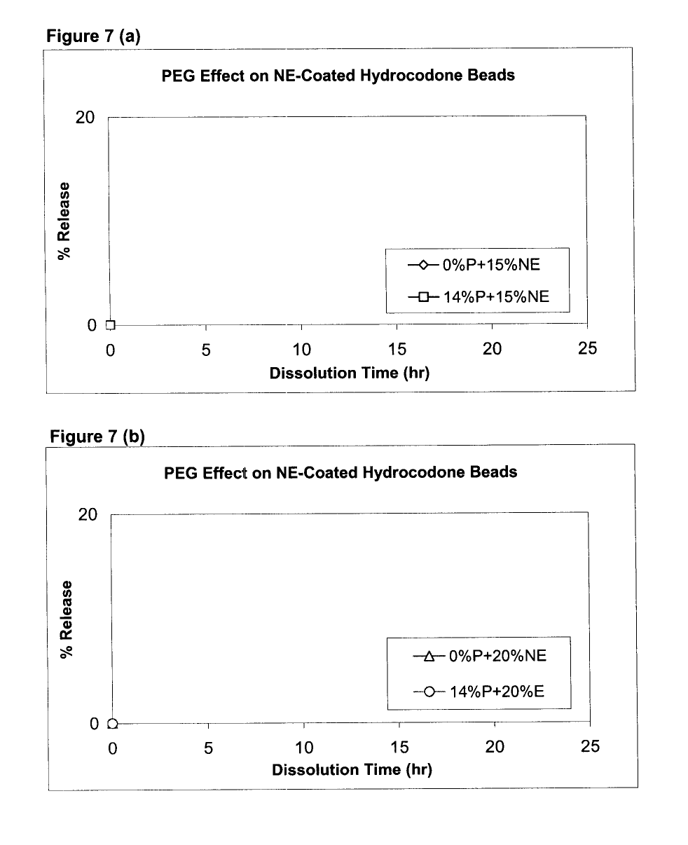

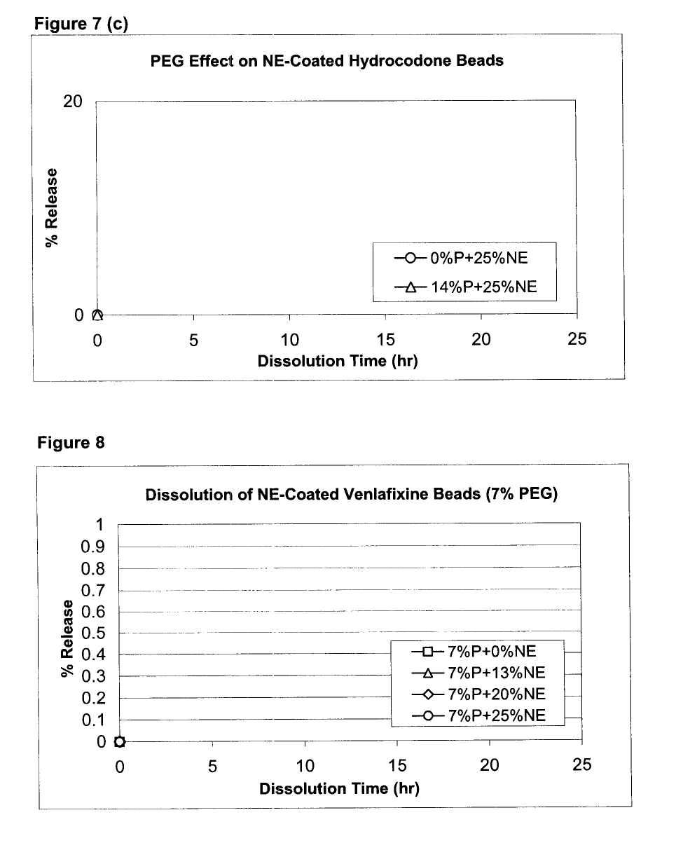

In FIG. 4, an increase in PEG surprisingly allowed for an increased amount of drug release. FIGS. 5-7 demonstrated similar results according to the invention as in FIGS. 1-4. FIG. 5 showed the use of increasing amounts of Eudragit® NE 30D coating allowed for increased control over the drug release. FIG. 6 illustrated that, with a constant amount of PEG, an increasing amount of polymer coating resulted in a desirable delay in release but at the expense of a decrease in cumulative hydrocodone release. In FIGS. 7a, 7b, and 7c, the dissolution profiles of hydrocodone were surprisingly regulated via Eudragit® NE 30D coating levels, while the impregnated PEG exhibited little effectiveness in controlling the rate of release.

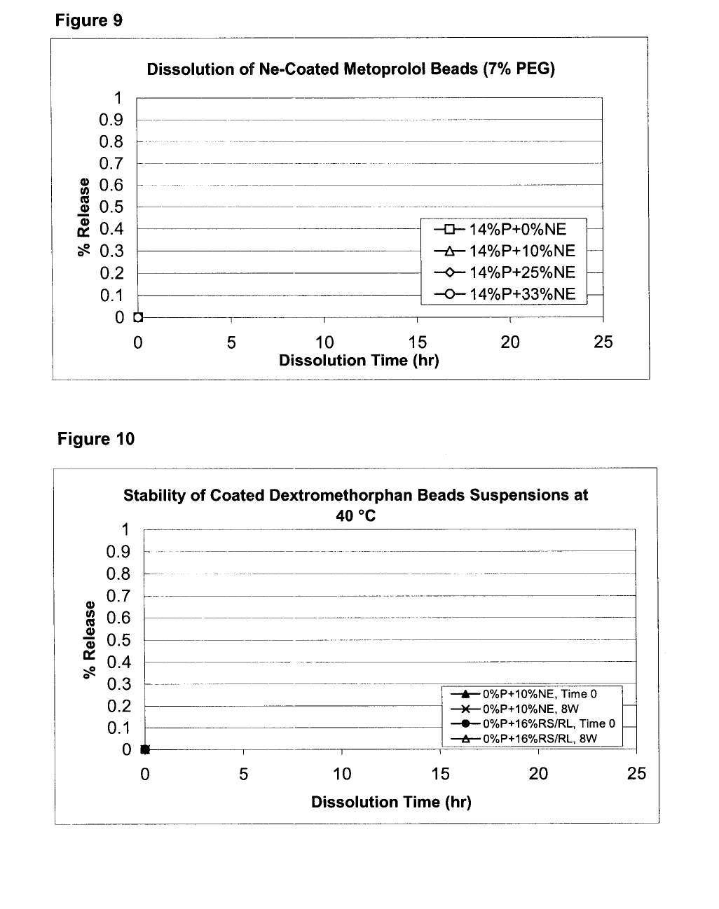

FIGS. 8 and 9 provide further dissolution data. FIG. 8 illustrated that, with a constant amount of PEG, an increasing amount of polymer coating resulted in a desirable delay in release of venlafaxine. FIG. 9 similarly illustrated that, with a constant amount of PEG, an increasing amount of polymer coating resulted in a desirable delay in release of metoprolol.

### Example 10

### Stability Data

Dissolution of stability samples (made according to Example 6) for 8 weeks at 40° C. is shown in FIG. 10. The data shows that there was little change in 10 weight percent Eudragit® NE coated dextromethorphan beads while 16 weight percent Eudragit® RS/RL coated beads showed incomplete release at time zero and a significantly decreased release rate at 8 weeks. The dissolution profile of the RS/RL changed significantly over time, which indicated the lack of stability of beads coated by this type of polymer. The data surprisingly and unexpectedly demonstrated the superior performance of Eudragit® NE coated beads of the invention over Eudragit® RS/RL coated ones, both when tested immediately and after 8 weeks in vitro.

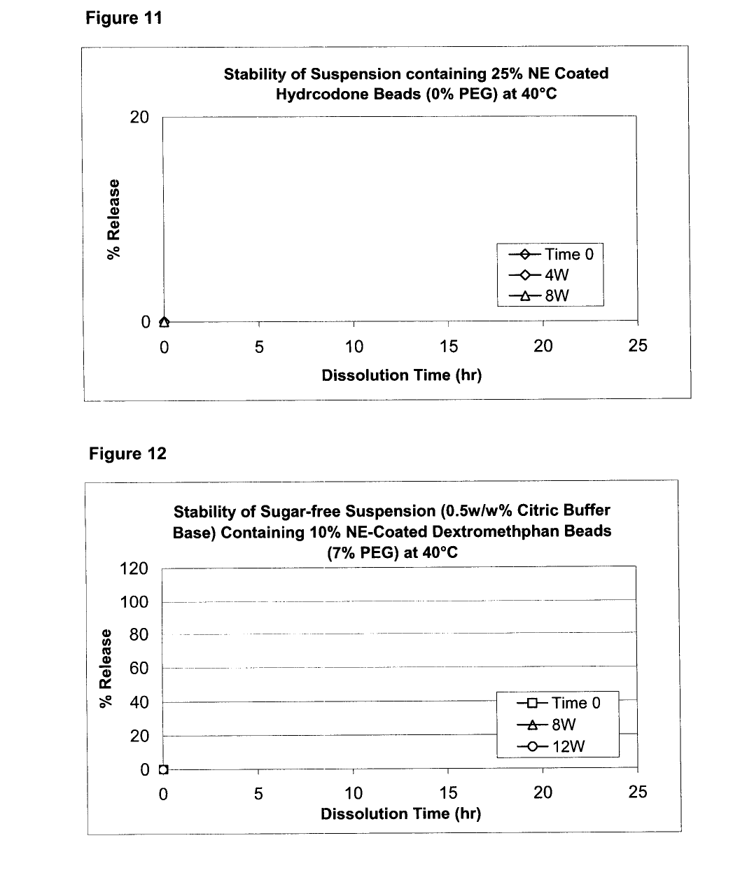

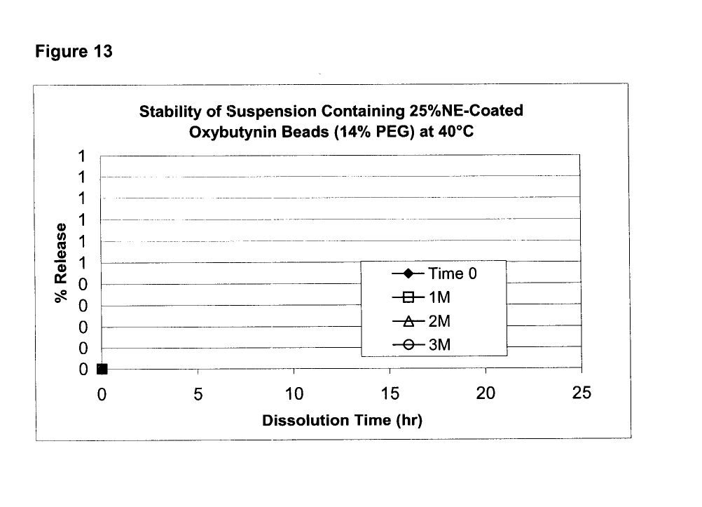

FIGS. 11 and 13 provide stability studies for polymer coated resin beads with various active ingredients. FIG. 11 illustrates stability in the hydrocodone dissolution over 4 and 8 weeks. FIG. 13 illustrates the stability of oxybutynin ion exchange resins in suspension over a three month period.

Two types of suspensions, in accordance with Example 6, were produced having varying citric acid concentrations. In Suspension I, the concentration of citric acid was 0.15% w/v. In Suspension II, the concentration of citric acid was 0.5% w/v. An amount of 22.9 g aqueous Eudragit NE®-coated dextromethorphan polistirex (7% PEG, 10% NE coating, drug loading is equal to 26.2%) was added into 1 liter well-mixed suspensions base to make dextromethorphan hydrobromide in the amount of 30 mg per 5 ml of suspension. The suspension was placed in an amber glass bottle and then placed into accelerated stability conditions at 40° C. for up to 12 weeks.

FIG. 12 illustrates the stability of Suspension II, which is Eudragit® NE coated dextromethorphan beads with 7% PEG loading suspended in a sugar-free vehicle in the presence of 0.5% w/v citric buffer. The dissolution of the suspension was relatively unchanged over the test period.

Stability data of Suspension I and II for 8 weeks are shown in Table 1. The data shows that there was little change in the dextromethorphan hydrobromide assay value in Suspension II for 8 weeks at 40° C. In contrast, in Suspension I, there was a drop in the dextromethorphan hydrobromide content, from 109.32% at time zero to 106.12% after 8 weeks. A citric acid concentration of 0.5 w/v % improved the dextromethorphan hydrobromide content by decreasing degradation under the accelerated stability conditions.

TABLE 1
Dextromethorphan Hydrobromide Content in Eudragit ® NE coated
Dextromethorphan Polistirex Prototype Suspension (Sugar free/EDTA
free) stability (Initial and 8 weeks at 40° C.)

| | Dextromethorphan | | | |
|---|---|---|---|---|
| | HBr Assay (% of | | Combined | |
| | Desired) | | Impurities (%)* | |
| Sample Description | 8 W, 40° C. | Initial | 8 W, 40° C. | Initial |
| Suspension I | 106.12 | 109.32 | 7.07 | 2.27 |
| Suspension II | 110.40 | 108.45 | 3.47 | 1.62 |

*The impurity level was calculated based on peak areas relative to that of the dextromethorpham HBr

Although preferred embodiments of the invention have been described in the foregoing description and figures, it will be understood that the invention is not limited to the specific embodiments disclosed herein but is capable of numerous modifications by one of ordinary skill in the art. It will be understood that the materials used and the chemical and pharmaceutical details may be slightly different or modified from the descriptions herein without departing from the methods and compositions disclosed and taught by the present invention.

What is claimed:

1. A method of making a coated drug/resin complex that comprises:
providing a drug/resin complex having an outer surface and comprising a plurality of resin beads and a therapeutically effective drug component releasably associated therewith; and
applying a coating layer comprising a methacrylate polymer over a portion of the outer surface of the complex in an amount sufficient to control the release rate of the drug component so as to form a coated drug/resin complex,
wherein the coating layer is at least substantially free of plasticizer and forms a flexible material that resists cracking.

2. The method of claim 1, wherein the methacrylate polymer is formed from an aqueous dispersion of a neutral copolymer comprising polyethyl acrylate and polymethyl methacrylate.

3. The method of claim 1, which further comprises suspending a therapeutically effective amount of the coated drug/resin complex in a pharmaceutically acceptable liquid carrier.

4. The method of claim 1, which further comprises associating a sufficient amount of polyethylene glycol with the drug/resin complex to facilitate eventual drug release.

5. The method of claim 3, which further comprises adding a stabilizing agent comprising one or more of a chelating agent, an antioxidant component, or both, in an amount sufficient to inhibit or prevent degradation of the coated drug/resin complex over a shelf-life of the composition.

6. The method of claim 5, wherein the stabilizing agent comprises citric acid or a salt thereof, ascorbic acid or a salt thereof, butylated hydroxyanisole, butylated hydroxytoluene, or any combination thereof.

7. The method of claim 1, wherein the applying is conducted at a temperature below a minimum film formation temperature.

8. The method of claim 7, wherein the minimum film formation temperature is below about 30° C.

9. The method of claim 1, further comprising adding a sufficient amount of an anti-tacking agent to minimize or prevent tackiness of the methacrylate polymer during the applying.

10. The method of claim 1, wherein the drug component is selected to comprise: hydrocodone, oxybutynin, dextromethorphan, fluoxetine, metoprolol, venlafaxine, buproprion, doxycycline, or verapamil, or a salt thereof, or any combination thereof.

11. The method of claim 4, wherein the polyethylene glycol is selected from a solid grade polyethylene glycol having a density of about 1.080 g/cm³ and a freezing point from about 50° C. to 62° C.

12. The method of claim 11, wherein the amount of polyethylene glycol is selected to be greater than about 5 weight percent of the drug/resin complex.

13. The method of claim 1, wherein the coating layer is disposed substantially entirely around each of a plurality of the resin beads.

14. A coated drug/resin complex that comprises:
a plurality of dry ion exchange resin forms complexed with a drug component to form a drug/resin complex; and
a substantially plasticizer-free coating layer having a thickness that comprises a sufficient amount of methacrylate polymer applied at least substantially around a portion of the resin forms to control the release rate of the drug component associated therewith.

15. The complex of claim 14, wherein the methacrylate polymer is formed from an aqueous dispersion of a neutral copolymer comprising polyethyl acrylate and polymethyl methacrylate.

16. The complex of claim 14, wherein the ratio of polyethyl acrylate to polymethyl methacrylate is about 0.5:1 to 10:1.

17. The complex of claim 14, which further comprises a sufficient amount of polyethylene glycol associated with the drug/resin complexes to facilitate drug release.

18. The complex of claim 14, wherein the sufficient amount of the methacrylate coating is greater than about 5 weight percent based on the coated drug/resin complex.

19. The complex of claim 18, wherein the sufficient amount is between about 7 weight percent to 40 weight percent based on the coated drug/resin complex.

20. The complex of claim 14, wherein the drug component comprises doxycycline, hydrocodone, metoprolol, fluoxetine, oxybutynin, dextromethorphan, bupropion, venlafaxine, or verapamil, or any salt thereof, or a combination thereof.

21. The complex of claim 17, wherein the amount of PEG is greater than about 5 weight percent based on the PEG-loaded drug/resin complex.

22. A pharmaceutical formulation comprising the complex of claim 14 and a pharmaceutically acceptable aqueous carrier and a stabilizing agent disposed therein in an amount sufficient to inhibit or prevent degradation of the coated drug/resin forms over a shelf life of the formulation.

23. The formulation of claim 22, wherein the aqueous carrier further comprises a buffering agent along with a suspending agent, a thickening agent, an anti-tacking agent, a preservative component, a sweetening agent, a mouth-feel agent, a wetting agent, or a combination thereof.

24. The formulation of claim 23, wherein the suspending agent is present and comprises HPMC, xanthan gum, microcrystalline cellulose, or a combination thereof; wherein the sweetener is present and comprises sucralose, acesulfame potassium, saccharin sodium, xylitol, liquid sugar, high-fructose corn syrup, or a combination thereof; wherein the preservative is present and comprises methyl paraben, propyl paraben, sodium benzoate, sodium metabisulfite, potassium sorbate, or a combination thereof; wherein the thickening agent is present and comprises starch; wherein the mouth feel agent is present and comprises glycerin; wherein the wetting agent is present and comprises propylene glycol; wherein the anti-tacking agent is present and comprises glyceryl monostearate and/or talc; or any combination thereof.

25. A coated drug/resin complex that comprises:
a plurality of dry ion exchange resin forms complexed with a drug component to form a drug/resin complex; and
a neutrally charged coating layer having a thickness that comprises a sufficient amount of methacrylate polymer applied at least substantially around a portion of the resin forms to control the release rate of the drug component associated therewith.

26. A method of making the coated drug/resin complex of claim 25 which comprises:
providing a drug/resin complex having an outer surface and comprising a plurality of resin beads and a therapeutically effective drug component releasably associated therewith; and
applying a coating layer comprising a methacrylate polymer over a portion of the outer surface of the complex in an amount sufficient to control the release rate of the drug component so as to form a coated drug/resin complex,
wherein the coating layer is neutral and forms a material that is sufficiently flexible to resist or avoid cracking.
